(19) United States
(12) Patent Application Publication
(10) Pub. No.: US 2025/0375392 A1
(43) Pub. Date: Dec. 11, 2025

(54) Branched Tail Lipids and Uses Thereof

(71) Applicant: Carnegie Mellon University, Pittsburgh, PA (US)

(72) Inventors: Kathryn Whitehead, Pittsburgh, PA (US); Mariah Lynn Arral, Pittsburgh, PA (US)

(21) Appl. No.: 19/232,612

(22) Filed: Jun. 9, 2025

Related U.S. Application Data
(60) Provisional application No. 63/657,224, filed on Jun. 7, 2024.

(51) Int. Cl. A61K 9/51; A61K 31/7105; A61K 31/713; A61K 47/28

(52) U.S. Cl. CPC A61K 9/5146; A61K 9/5123; A61K 31/7105; A61K 31/713; A61K 47/28

(57) ABSTRACT

Ionizable lipids having branched tails, nanoparticles containing the ionizable lipids, compositions containing the nanoparticles, and methods for using the ionizable lipids, nanoparticles, and compositions to deliver agents (e.g., RNAs) to cells, tissues, and/or organs are provided herein.

### CROSS-REFERENCE TO RELATED APPLICATIONS

This application claims priority from U.S. Provisional Application Ser. No. 63/657,224, filed Jun. 7, 2024. The disclosure of the prior application is considered part of (and is incorporated by reference in) the disclosure of this application.

### TECHNICAL FIELD

This document relates to ionizable lipids having branched tails, nanoparticles containing the ionizable lipids, compositions containing the nanoparticles, and methods for using the ionizable lipids, nanoparticles, and compositions to deliver agents (e.g., RNAs) to cells, tissues, and/or organs.

### BACKGROUND

Lipid nanoparticles are the leading-edge technology for mRNA therapeutics, serving as vehicles for delivering mRNA to target cells since naked mRNA is immunogenic and inefficacious (Karikó et al., Immunity(2005), 23:165-175; Hajj et al., Small(2019), 15(6), doi.org/10.1002/smll.201805097; and Karikó et al., Mol Ther(2008), 16:1833-1840). Lipid nanoparticle mRNA therapeutics have the potential to treat or prevent diseases such as cancer (Billingsley et al., Nano Lett(2020), 20(3): 1578-1589(2020); Oberli et al., Nano Lett(2017), 17:1326-1335; and Liu et al., J Contr Rel(2022)345:306-313), infectious diseases, and genetic disorders (Han et al., Sci Adv(2022), 8:6901; and Witzigmann et al., Adv Drug Deliv Rev(2020), 159:344-363). In addition to the clinical success of the SARS-COV-2 mRNA vaccines by Moderna (Corbett et al., New Engl J Med(2020), 383:1544-1555; and Anderson et al., New Engl J Med(2020), 383:2427-2438) and BioNTech/Pfizer (Polack et al., New Engl J Med(2020), 383:2603-2615; and Barda et al., New Engl J Med(2021), 385:1078-1090), clinical trials have incorporated lipid nanoparticles for treatment of diseases such as transthyretin amyloidosis (see, e.g., ir.intelliatx.com/press-releases) and for protection against infectious diseases such as malaria (see, e.g., Tsoumani et al., Vaccines(Basel) (2023), 11(9): 1452).

Most lipid nanoparticles contain four components: an ionizable lipid (also referred to as a lipidoid), a helper lipid, a sterol, and a polyethylene glycol-(PEG-) lipid or PEG-cholesterol conjugate (LoPresti et al., J Contr Rel(2022), 345:819-831). The helper lipid is a phospholipid, such as DSPC or DOPE, that assists the lipid nanoparticle with cell entry and endosomal fusion (Eygeris et al., Acc Chem Res (2022), 55:2-12). Cholesterol (Patel et al., Nat Commun(2020), 11:1-13; and Paunovska et al., Adv Mat(2019), 31:1807748) is the most ubiquitously used sterol in lipid nanoparticle formulations, while PEG-lipids can differ in length and linker depending on the application (Ryals et al., PLOS One(2020), 15: e0241006; Lokugamage et al., Nature Biomed Eng (2021), 5:1059-1068; and Hatakeyama et al., Biomaterials(2011), 32:4306-4316). Ionizable lipids are non-natural lipids that have been synthesized by academic and industrial laboratories. Ionizable lipids typically feature an ionizable amine-containing headgroup, hydrophobic tails, and a biodegradable linker embedded within the tails (Chen et al., J Am Chem Soc(2023), 145:24302-24314). The headgroup aids in mRNA condensation during nanoparticle formation and also aids in interaction with anionic phospholipids in cellular and endosomal membranes, facilitating cell entry and escape (Chen et al., supra). In some cases, the hydrophobic tails of an ionizable lipid can be branched. Branched tails can adopt a cone-shaped conformation; ionizable lipids with a cone shape can adopt an inverted hexagonal conformation in acidic environments such as the endosome. This conformation can aid in endosomal membrane fusion (Semple et al., Nature Biotechnol(2010), 28:172-176; and Koltover et al., Science(1998), 281:78-81). However, only a small number of branched tail lipids have been investigated. As such, there is an inadequate understanding of their structure-function relationships as well as a limited pool of materials available for clinical translation.

### SUMMARY

Ionizable lipids are lipids that, when incorporated into a lipid nanoparticle (LNP), result in a LNP having a slightly negative or neutral charge at physiological pH (7.0) and a positive charge at endosomal pH (4.5-6.5). As described herein, a small library of ionizable lipids having branched hydrophobic tails was generated for incorporation into lipid nanoparticles, and the efficacy of the resulting lipid nanoparticles was examined. This document is based, at least in part, on the identification of novel branched tail lipidoids and their incorporation into nanoparticles effective to deliver nucleic acid (e.g., RNA) to cells. As described herein, branched tail ionizable lipids were synthesized, characterized, and screened in vivo to better understand how the branching point and carbon chain length can impact delivery outcomes. The in vivo screen led to the identification of new ionizable branched tail lipids that were at least as potent as the “gold standard” or “benchmark,” 306O i10(also referred to herein as 10(8)1 to convey branching structure). The efficacy of the branched tail lipid nanoparticles was correlated with increased ionization at endosomal pH and pKa values. The branched tail lipid nanoparticles outperformed their unbranched, similar lipid tail length counterparts between 3 and 18-fold on average. Interestingly, in some cases the organ tropism did not shift with a change in branching. The branched tail lipid nanoparticles were investigated further for their versatility in mRNA delivery. These branched tail lipid nanoparticles were effective to deliver three mRNAs simultaneously and to co-deliver messenger RNA (mRNA) and small interfering RNA (siRNA). Further, they were efficacious across different administration routes.

This document provides ionizable lipids, lipid nanoparticles containing the ionizable lipids, and methods for their use (e.g., for drug delivery). In some cases, the ionizable lipids described herein include an acrylate tail and a novel carbon backbone. Such lipids can be made by reacting an amine-containing head and acrylate tails through a Michael addition reaction, during which primary amine hydrogens on the heads are substituted with the acrylate tail. The lipids provided herein can be used, for example, to generate LNPs. In some cases, LNPs described herein can be formulated with therapeutic agents (e.g., nucleic acids including, but not limited to, mRNA and/or siRNA), and can deliver that cargo into the cytoplasm of target cells. This document also provides effective delivery vehicles. In some cases, the methods and materials described herein can be used to unlock treatments for diseases (including, but not limited to, diseases in the liver and/or spleen).

In a first aspect, this document features a lipid-containing particle. The lipid-containing particle can include, consist of, or consist essentially of a lipidoid, cholesterol or a derivative thereof, a helper lipid, and a polyethylene glycol (PEG)-based compound, where the said lipidoid includes an amine-containing head and one or more acrylate tails, where each of the aid one or more acrylate tails has an alkyl chain of 10 or 11 carbon atoms, and where the alkyl chain of each of the one or more acrylate tails has a one, two, three, four, or five carbon atom branch. The lipidoid can have two, three, or four acrylate tails. Each of the acrylate tails can be the same, or the lipidoid can include two or more different acrylate tails. The lipid-containing particle can be a lipid nanoparticle (LNP). The helper lipid is a neutral lipid or a zwitterionic lipid. The neutral or zwitterionic helper lipid can include one or more of 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-distearoyl-sn-glycero-3-phosphoethanolamine (DSPE), 1,2-dipalmitoyl-sn-glycero-3-phosphocholine (DPPC), 1-palmitoyl-2-oleoyl-sn-glycero-3-phosphocholine (POPC), 1,2-dimyristoyl-sn-glycero-3-phosphocholine (DMPC), 1,2-dipalmitoyl-sn-glycero-3-phosphoethanolamine (DPPE), 1-palmitoyl-2-oleoyl-sn-glycero-3-phosphoethanolamine (POPE), 1,2-di-myristoyl-sn-glycero-3-phosphoethanolamine (DMPE), palmitoyl sphingomyelin (PSM), sterol sphingomyelin (SSM), a triglyceride, a triacylglycerol, a diglyceride, a diacylglycerol, and a ceramide. The helper lipid can be a cationic lipid. The cationic helper lipid can include one or more of 1,2-dileoyl-3-trimethylammonium-propane (DOTAP), 1,2-dimyristoyl-sn-glycero-3-trimethylammonium-propane (DMTAP), 1,2 dipalmitoyl-sn-glycero-3-trimethylammoniumpropane (DPTAP), 1,2-distearoyl-sn-glycero-3-trimethylammoniumpropane (DSTAP), 3β-[N-(N′,N′-dimethylaminoethane)-carbamoyl]cholesterol (DC-cholesterol), didodecyldimethylammonium bromide (DDAB), dioctadecyloxy-propyl-glycerol (DOGS), dimethyldioctadecylammonium bromide (DODAB), dioleyloxy-propyl-trimethylammonium (DOSPA), and N-[1-(2,3-dioleoyloxy) propyl]-N,N,N-trimethylammonium chloride (DOTMA). The lipid-containing particle can be liver-tropic. The lipid-containing particle can be spleen-tropic. The lipidoid can include an N¹-(3-aminopropyl)-N¹-methylpropane-1,3-diamine head. The lipidoid can include a decan-2-yl acrylate (10(1)1) tail, a decan-3-yl acrylate (10(1)2) tail, a decan-4-yl acrylate (10(1)3) tail, a decan-5-yl acrylate (10(1)4) tail, a 2-methylnonyl acrylate (10(2)1) tail, a 2-ethyloctyl acrylate (10(2)2) tail, a 2-propylheptyl acrylate (10(2)3) tail, a 2-butylhexyl acrylate (10(2)4) tail, a 3-methylnonyl acrylate (10(3)1) tail, a 3-ethyloctyl acrylate (10(3)2) tail, a 3-propylheptyl acrylate (10(3)3) tail, a 4-methylnonyl acrylate (10(4)1) tail, a 4-ethyloctyl acrylate (10(4)2) tail, a 4-propylheptyl acrylate (10(4)3) tail, a 5-methynonyl acrylate (10(5)1) tail, a 6-methylnonyl acrylate (10(6)1) tail, or a 6-ethyloctyl acrylate (10(6)2) tail. The lipidoid can include an undecan-3-yl acrylate (11(1)2) tail, an undecan-4-yl acrylate (11(1)3) tail, an undecan-5-yl acrylate (11(1)4) tail, an undecan-6-yl acrylate (11(1)5) tail, a 7-ethylnonyl acrylate (11(7)2) tail, an 8-methyldecyl acrylate (11(8)1) tail, or a 9-methyldecyl acrylate (11(9)1) tail. The cholesterol or derivative thereof can be cholesterol. The PEG-based compound can be a PEG-lipid (e.g., where the PEG has a molecular weight of about 300 g/mol to about 5000 g/mol). At pH 5.5, the lipid-containing particle can have a net positive charge. The lipid-containing particle can have a diameter of about 60 nM to about 160 nM. The lipid-containing particle can have a zeta potential at pH 7.4 of about −11 mV to about −2 mV. The lipid-containing particle can have a pKa of about 5.3 to about 7.5. In some cases, the branch is not at the second carbon. In some cases, the branch can be at the first, third, fourth, fifth, or sixth carbon.

In another aspect, this document features a composition containing, consisting of, or consisting essentially of a lipid-containing particle, where the lipid-containing particle includes: a lipidoid having an amine-containing head and one or more acrylate tails, wherein each of the one or more acrylate tails includes an alkyl chain of 10 or 11 carbon atoms, and where the alkyl chain of each of the one or more acrylate tails has a one, two, three, four, or five carbon atom branch; cholesterol or a derivative thereof; a helper lipid; a PEG-based compound; and a therapeutic agent. The therapeutic agent can include an RNA. The RNA can include mRNA, short interfering RNA (siRNA), short hairpin RNA (shRNA), micro RNA (miRNA), antisense RNA, guide RNA, long non-coding RNA, transfer RNA, ribosomal RNA, double-stranded RNA (dsRNA), RNA aptamer, or any combination thereof. The lipid-containing particle can include two or more mRNA molecules and one or more siRNA molecules. In some cases, the lipid-containing particle can include two mRNA molecules, where a first of the two mRNA molecules encodes a light chain of an antibody, and wherein a second of the two mRNA molecules encodes a heavy chain of the antibody. The lipidoid can have two, three, or four acrylate tails. Each of the acrylate tails can be the same, or the lipidoid can include two or more different acrylate tails. The lipid-containing particle can be a lipid nanoparticle (LNP). The helper lipid is a neutral lipid or a zwitterionic lipid. The neutral or zwitterionic helper lipid can include one or more of DSPC, DOPE, DOPC, DSPE, DPPC, POPC, DMPC, DPPE, POPE, DMPE, PSM, SSM, a triglyceride, a triacylglycerol, a diglyceride, a diacylglycerol, and a ceramide. The helper lipid can be a cationic lipid. The cationic helper lipid can include one or more of DOTAP, DMTAP, DPTAP, DSTAP, DC-cholesterol, DDAB, DOGS, DODAB, DOSPA, and DOTMA. The lipid-containing particle can be liver-tropic. The lipid-containing particle can be spleen-tropic. The lipidoid can include an N¹-(3-aminopropyl)-N¹-methylpropane-1,3-diamine head. The lipidoid can include a decan-2-yl acrylate (10(1)1) tail, a decan-3-yl acrylate (10(1)2) tail, a decan-4-yl acrylate (10(1)3) tail, a decan-5-yl acrylate (10(1)4) tail, a 2-methylnonyl acrylate (10(2)1) tail, a 2-ethyloctyl acrylate (10(2)2) tail, a 2-propylheptyl acrylate (10(2)3) tail, a 2-butylhexyl acrylate (10(2)4) tail, a 3-methylnonyl acrylate (10(3)1) tail, a 3-ethyloctyl acrylate (10(3)2) tail, a 3-propylheptyl acrylate (10(3)3) tail, a 4-methylnonyl acrylate (10(4)1) tail, a 4-ethyloctyl acrylate (10(4)2) tail, a 4-propylheptyl acrylate (10(4)3) tail, a 5-methynonyl acrylate (10(5)1) tail, a 6-methylnonyl acrylate (10(6)1) tail, or a 6-ethyloctyl acrylate (10(6)2) tail. The lipidoid can include an undecan-3-yl acrylate (11(1)2) tail, an undecan-4-yl acrylate (11(1)3) tail, an undecan-5-yl acrylate (11(1)4) tail, an undecan-6-yl acrylate (11(1)5) tail, a 7-ethylnonyl acrylate (11(7)2) tail, an 8-methyldecyl acrylate (11(8)1) tail, or a 9-methyldecyl acrylate (11(9)1) tail. The cholesterol or derivative thereof can be cholesterol. The PEG-based compound can be a PEG-lipid (e.g., where the PEG has a molecular weight of about 300 g/mol to about 5000 g/mol). At pH 5.5, the lipid-containing particle can have a net positive charge. The lipid-containing particle can have a diameter of about 60 nM to about 160 nM. The lipid-containing particle can have a zeta potential at pH 7.4 of about −11 mV to about −2 mV. The lipid-containing particle can have a pKa of about 5.3 to about 7.5. In some cases, the branch is not at the second carbon. In some cases, the branch can be at the first, third, fourth, fifth, or sixth carbon.

In another aspect, this document features a method for delivering a therapeutic agent to a mammal. The method can include, consist of, or consist essentially of administering to the mammal a composition containing a lipid-containing particle, where the lipid-containing particle includes: a lipidoid having an amine-containing head and one or more acrylate tails, where each of the one or more acrylate tails includes an alkyl chain of 10 or 11 carbon atoms, and where the alkyl chain of each of the one or more acrylate tails has a one, two, three, four, or five carbon atom branch; cholesterol or a derivative thereof; a helper lipid; a PEG-based compound; and the therapeutic agent. The therapeutic agent can include an RNA. The RNA can include mRNA, siRNA, shRNA, miRNA, antisense RNA, guide RNA, long non-coding RNA, transfer RNA, ribosomal RNA, dsRNA, RNA aptamer, or any combination thereof. The lipid-containing particle can include two or more mRNA molecules and one or more siRNA molecules. In some cases, the lipid-containing particle can include two mRNA molecules, where a first of the two mRNA molecules encodes a light chain of an antibody, and where a second of the two mRNA molecules encodes a heavy chain of the antibody. The mammal can have been identified as having a liver disorder. The liver disorder can include alpha-1 antitrypsin deficiency, hereditary hemochromatosis, Wilson's disease, hereditary tyrosinemia, glycogen storage diseases, viral hepatitis, familial hypercholesterolemia, nonalcoholic fatty liver disease, primary hyperoxaluria, acute intermittent porphyria, hepatocellular carcinoma, paroxysmal nocturnal hemoglobinuria, or any combination thereof. The mammal can have been identified as having COVID-19, Rift Valley Fever Virus, cancer, or any combination thereof. The mammal can be a human. The lipidoid can have two, three, or four acrylate tails. Each of the acrylate tails can be the same, or the lipidoid can include two or more different acrylate tails. The lipid-containing particle can be a lipid nanoparticle (LNP). The helper lipid is a neutral lipid or a zwitterionic lipid. The neutral or zwitterionic helper lipid can include one or more of DSPC, DOPE, DOPC, DSPE, DPPC, POPC, DMPC, DPPE, POPE, DMPE, PSM, SSM, a triglyceride, a triacylglycerol, a diglyceride, a diacylglycerol, and a ceramide. The helper lipid can be a cationic lipid. The cationic helper lipid can include one or more of DOTAP, DMTAP, DPTAP, DSTAP, DC-cholesterol, DDAB, DOGS, DODAB, DOSPA, and DOTMA. The lipid-containing particle can be liver-tropic. The lipid-containing particle can be spleen-tropic. The lipidoid can include an N¹-(3-aminopropyl)-N¹-methylpropane-1,3-diamine head. The lipidoid can include a decan-2-yl acrylate (10(1)1) tail, a decan-3-yl acrylate (10(1)2) tail, a decan-4-yl acrylate (10(1)3) tail, a decan-5-yl acrylate (10(1)4) tail, a 2-methylnonyl acrylate (10(2)1) tail, a 2-ethyloctyl acrylate (10(2)2) tail, a 2-propylheptyl acrylate (10(2)3) tail, a 2-butylhexyl acrylate (10(2)4) tail, a 3-methylnonyl acrylate (10(3)1) tail, a 3-ethyloctyl acrylate (10(3)2) tail, a 3-propylheptyl acrylate (10(3)3) tail, a 4-methylnonyl acrylate (10(4)1) tail, a 4-ethyloctyl acrylate (10(4)2) tail, a 4-propylheptyl acrylate (10(4)3) tail, a 5-methynonyl acrylate (10(5)1) tail, a 6-methylnonyl acrylate (10(6)1) tail, or a 6-ethyloctyl acrylate (10(6)2) tail. The lipidoid can include an undecan-3-yl acrylate (11(1)2) tail, an undecan-4-yl acrylate (11(1)3) tail, an undecan-5-yl acrylate (11(1)4) tail, an undecan-6-yl acrylate (11(1)5) tail, a 7-ethylnonyl acrylate (11(7)2) tail, an 8-methyldecyl acrylate (11(8)1) tail, or a 9-methyldecyl acrylate (11(9)1) tail. The cholesterol or derivative thereof can be cholesterol. The PEG-based compound can be a PEG-lipid (e.g., where the PEG has a molecular weight of about 300 g/mol to about 5000 g/mol). At pH 5.5, the lipid-containing particle can have a net positive charge. The lipid-containing particle can have a diameter of about 60 nM to about 160 nM. The lipid-containing particle can have a zeta potential at pH 7.4 of about −11 mV to about −2 mV. The lipid-containing particle can have a pKa of about 5.3 to about 7.5. In some cases, the branch is not at the second carbon. In some cases, the branch can be at the first, third, fourth, fifth, or sixth carbon.

In another aspect, this document features a lipid-containing particle, where the lipid-containing particle includes, consists of, or consists essentially of: a lipidoid; cholesterol or a derivative thereof; a helper lipid; and a PEG-based compound, where the lipidoid includes an amine-containing head and one or more acrylate tails, where the amine-containing head is N¹-(3-aminopropyl)-N¹-methylpropane-1,3-diamine, and where the one or more acrylate tails include one or more of a decan-2-yl acrylate (10(1)1) tail, a decan-3-yl acrylate (10(1)2) tail, a decan-4-yl acrylate (10(1)3) tail, a decan-5-yl acrylate (10(1)4) tail, a 2-methylnonyl acrylate (10(2)1) tail, a 2-ethyloctyl acrylate (10(2)2) tail, a 2-propylheptyl acrylate (10(2)3) tail, a 2-butylhexyl acrylate (10(2)4) tail, a 3-methylnonyl acrylate (10(3)1) tail, a 3-ethyloctyl acrylate (10(3)2) tail, a 3-propylheptyl acrylate (10(3)3) tail, a 4-methylnonyl acrylate (10(4)1) tail, a 4-ethyloctyl acrylate (10(4)2) tail, a 4-propylheptyl acrylate (10(4)3) tail, a 5-methynonyl acrylate (10(5)1) tail, a 6-methylnonyl acrylate (10(6)1) tail, a 6-ethyloctyl acrylate (10(6)2) tail, an undecan-3-yl acrylate (11(1)2) tail, an undecan-4-yl acrylate (11(1)3) tail, an undecan-5-yl acrylate (11(1)4) tail, an undecan-6-yl acrylate (11(1)5) tail, a 7-ethylnonyl acrylate (11(7)2) tail, an 8-methyldecyl acrylate (11(8)1) tail, and a 9-methyldecyl acrylate (11(9)1) tail.

In another aspect, this document features a composition containing a lipid-containing particle that includes a therapeutic agent, where the lipid-containing particle includes, consists of, or consists essentially of: a lipidoid; cholesterol or a derivative thereof; a helper lipid; and a PEG-based compound, where the lipidoid includes an amine-containing head and one or more acrylate tails, where the amine-containing head is N¹-(3-aminopropyl)-N¹-methylpropane-1,3-diamine, and where the one or more acrylate tails include one or more of a decan-2-yl acrylate (10(1)1) tail, a decan-3-yl acrylate (10(1)2) tail, a decan-4-yl acrylate (10(1)3) tail, a decan-5-yl acrylate (10(1)4) tail, a 2-methylnonyl acrylate (10(2)1) tail, a 2-ethyloctyl acrylate (10(2)2) tail, a 2-propylheptyl acrylate (10(2)3) tail, a 2-butylhexyl acrylate (10(2)4) tail, a 3-methylnonyl acrylate (10(3)1) tail, a 3-ethyloctyl acrylate (10(3)2) tail, a 3-propylheptyl acrylate (10(3)3) tail, a 4-methylnonyl acrylate (10(4)1) tail, a 4-ethyloctyl acrylate (10(4)2) tail, a 4-propylheptyl acrylate (10(4)3) tail, a 5-methynonyl acrylate (10(5)1) tail, a 6-methylnonyl acrylate (10(6)1) tail, a 6-ethyloctyl acrylate (10(6)2) tail, an undecan-3-yl acrylate (11(1)2) tail, an undecan-4-yl acrylate (11(1)3) tail, an undecan-5-yl acrylate (11(1)4) tail, an undecan-6-yl acrylate (11(1)5) tail, a 7-ethylnonyl acrylate (11(7)2) tail, an 8-methyldecyl acrylate (11(8)1) tail, and a 9-methyldecyl acrylate (11(9)1) tail. The therapeutic agent can include RNA. The RNA can include mRNA, siRNA, shRNA, miRNA, antisense RNA, guide RNA, long non-coding RNA, transfer RNA, ribosomal RNA, dsRNA, an RNA aptamer, or any combination thereof. The lipid-containing particle can include two or more mRNA molecules and one or more siRNA molecules. In some cases, the lipid-containing particle can include two mRNA molecules, wherein a first of said two mRNA molecules encodes a light chain of an antibody, and wherein a second of said two mRNA molecules encodes a heavy chain of the antibody. The lipidoid can have two, three, or four acrylate tails. Each of the acrylate tails can be the same, or the lipidoid can include two or more different acrylate tails. The lipid-containing particle can be a lipid nanoparticle (LNP). The helper lipid is a neutral lipid or a zwitterionic lipid. The neutral or zwitterionic helper lipid can include one or more of DSPC, DOPE, DOPC, DSPE, DPPC, POPC, DMPC, DPPE, POPE, DMPE, PSM, SSM, a triglyceride, a triacylglycerol, a diglyceride, a diacylglycerol, and a ceramide. The helper lipid can be a cationic lipid. The cationic helper lipid can include one or more of DOTAP, DMTAP, DPTAP, DSTAP, DC-cholesterol, DDAB, DOGS, DODAB, DOSPA, and DOTMA. The lipid-containing particle can be liver-tropic. The lipid-containing particle can be spleen-tropic. The cholesterol or derivative thereof can be cholesterol. The PEG-based compound can be a PEG-lipid (e.g., where the PEG has a molecular weight of about 300 g/mol to about 5000 g/mol). At pH 5.5, the lipid-containing particle can have a net positive charge. The lipid-containing particle can have a diameter of about 60 nM to about 160 nM. The lipid-containing particle can have a zeta potential at pH 7.4 of about −11 mV to about −2 mV. The lipid-containing particle can have a pKa of about 5.3 to about 7.5.

In another aspect, this document features a method for delivering a therapeutic agent to a mammal. The method can include, consist of, or consist essentially of administering to the mammal a composition containing a lipid-containing particle that includes the therapeutic agent, where the lipid-containing particle includes, consists of, or consists essentially of: a lipidoid; cholesterol or a derivative thereof; a helper lipid; and a PEG-based compound, where the lipidoid includes an amine-containing head and one or more acrylate tails, where the amine-containing head is N¹-(3-aminopropyl)-N¹-methylpropane-1,3-diamine, and where the one or more acrylate tails include one or more of a decan-2-yl acrylate (10(1)1) tail, a decan-3-yl acrylate (10(1)2) tail, a decan-4-yl acrylate (10(1)3) tail, a decan-5-yl acrylate (10(1)4) tail, a 2-methylnonyl acrylate (10(2)1) tail, a 2-ethyloctyl acrylate (10(2)2) tail, a 2-propylheptyl acrylate (10(2)3) tail, a 2-butylhexyl acrylate (10(2)4) tail, a 3-methylnonyl acrylate (10(3)1) tail, a 3-ethyloctyl acrylate (10(3)2) tail, a 3-propylheptyl acrylate (10(3)3) tail, a 4-methylnonyl acrylate (10(4)1) tail, a 4-ethyloctyl acrylate (10(4)2) tail, a 4-propylheptyl acrylate (10(4)3) tail, a 5-methynonyl acrylate (10(5)1) tail, a 6-methylnonyl acrylate (10(6)1) tail, a 6-ethyloctyl acrylate (10(6)2) tail, an undecan-3-yl acrylate (11(1)2) tail, an undecan-4-yl acrylate (11(1)3) tail, an undecan-5-yl acrylate (11(1)4) tail, an undecan-6-yl acrylate (11(1)5) tail, a 7-ethylnonyl acrylate (11(7)2) tail, an 8-methyldecyl acrylate (11(8)1) tail, and a 9-methyldecyl acrylate (11(9)1) tail. The therapeutic agent can include RNA. The RNA can include mRNA, siRNA, shRNA, miRNA, antisense RNA, guide RNA, long non-coding RNA, transfer RNA, ribosomal RNA, dsRNA, an RNA aptamer, or any combination thereof. The lipid-containing particle can include two or more mRNA molecules and one or more siRNA molecules. In some cases, the lipid-containing particle can include two mRNA molecules, wherein a first of the two mRNA molecules encodes a light chain of an antibody, and where a second of the two mRNA molecules encodes a heavy chain of the antibody. The mammal can have been identified as having a liver disorder. The liver disorder can include alpha-1 antitrypsin deficiency, hereditary hemochromatosis, Wilson's disease, hereditary tyrosinemia, glycogen storage diseases, viral hepatitis, familial hypercholesterolemia, nonalcoholic fatty liver disease, primary hyperoxaluria, acute intermittent porphyria, hepatocellular carcinoma, paroxysmal nocturnal hemoglobinuria, or any combination thereof. The mammal can have been identified as having COVID-19, Rift Valley Fever Virus, cancer, or any combination thereof. The mammal can be a human. The lipidoid can have two, three, or four acrylate tails. Each of the acrylate tails can be the same, or the lipidoid can include two or more different acrylate tails. The lipid-containing particle can be a lipid nanoparticle (LNP). The helper lipid is a neutral lipid or a zwitterionic lipid. The neutral or zwitterionic helper lipid can include one or more of DSPC, DOPE, DOPC, DSPE, DPPC, POPC, DMPC, DPPE, POPE, DMPE, PSM, SSM, a triglyceride, a triacylglycerol, a diglyceride, a diacylglycerol, and a ceramide. The helper lipid can be a cationic lipid. The cationic helper lipid can include one or more of DOTAP, DMTAP, DPTAP, DSTAP, DC-cholesterol, DDAB, DOGS, DODAB, DOSPA, and DOTMA. The lipid-containing particle can be liver-tropic. The lipid-containing particle can be spleen-tropic. The cholesterol or derivative thereof can be cholesterol. The PEG-based compound can be a PEG-lipid (e.g., where the PEG has a molecular weight of about 300 g/mol to about 5000 g/mol). At pH 5.5, the lipid-containing particle can have a net positive charge. The lipid-containing particle can have a diameter of about 60 nM to about 160 nM. The lipid-containing particle can have a zeta potential at pH 7.4 of about −11 mV to about −2 mV. The lipid-containing particle can have a pKa of about 5.3 to about 7.5.

In another aspect, this document features a method for treating a mammal having a liver disorder or a symptom thereof. The method can include, consist of, or consist essentially of administering to the mammal a composition containing a lipid-containing particle that includes a therapeutic agent, where the lipid-containing particle includes, consists of, or consists essentially of: a lipidoid; cholesterol or a derivative thereof; a helper lipid; and a PEG-based compound, where the lipidoid includes an amine-containing head and one or more acrylate tails, where the amine-containing head is N¹-(3-aminopropyl)-N¹-methylpropane-1,3-diamine, and where the one or more acrylate tails include one or more of a decan-2-yl acrylate (10(1)1) tail, a decan-3-yl acrylate (10(1)2) tail, a decan-4-yl acrylate (10(1)3) tail, a decan-5-yl acrylate (10(1)4) tail, a 2-methylnonyl acrylate (10(2)1) tail, a 2-ethyloctyl acrylate (10(2)2) tail, a 2-propylheptyl acrylate (10(2)3) tail, a 2-butylhexyl acrylate (10(2)4) tail, a 3-methylnonyl acrylate (10(3)1) tail, a 3-ethyloctyl acrylate (10(3)2) tail, a 3-propylheptyl acrylate (10(3)3) tail, a 4-methylnonyl acrylate (10(4)1) tail, a 4-ethyloctyl acrylate (10(4)2) tail, a 4-propylheptyl acrylate (10(4)3) tail, a 5-methynonyl acrylate (10(5)1) tail, a 6-methylnonyl acrylate (10(6)1) tail, a 6-ethyloctyl acrylate (10(6)2) tail, an undecan-3-yl acrylate (11(1)2) tail, an undecan-4-yl acrylate (11(1)3) tail, an undecan-5-yl acrylate (11(1)4) tail, an undecan-6-yl acrylate (11(1)5) tail, a 7-ethylnonyl acrylate (11(7)2) tail, an 8-methyldecyl acrylate (11(8)1) tail, and a 9-methyldecyl acrylate (11(9)1) tail. The therapeutic agent can include RNA. The RNA can include mRNA, siRNA, shRNA, miRNA, antisense RNA, guide RNA, long non-coding RNA, transfer RNA, ribosomal RNA, dsRNA, an RNA aptamer, or any combination thereof. The lipid-containing particle can include two or more mRNA molecules and one or more siRNA molecules. In some cases, the lipid-containing particle can include two mRNA molecules, where a first of the two mRNA molecules encodes a light chain of an antibody, and where a second of the two mRNA molecules encodes a heavy chain of the antibody. The liver disorder can include alpha-1 antitrypsin deficiency, hereditary hemochromatosis, Wilson's disease, hereditary tyrosinemia, glycogen storage diseases, viral hepatitis, familial hypercholesterolemia, nonalcoholic fatty liver disease, primary hyperoxaluria, acute intermittent porphyria, hepatocellular carcinoma, paroxysmal nocturnal hemoglobinuria, or any combination thereof. The mammal can be a human. The lipidoid can have two, three, or four acrylate tails. Each of the acrylate tails can be the same, or the lipidoid can include two or more different acrylate tails. The lipid-containing particle can be a lipid nanoparticle (LNP). The helper lipid is a neutral lipid or a zwitterionic lipid. The neutral or zwitterionic helper lipid can include one or more of DSPC, DOPE, DOPC, DSPE, DPPC, POPC, DMPC, DPPE, POPE, DMPE, PSM, SSM, a triglyceride, a triacylglycerol, a diglyceride, a diacylglycerol, and a ceramide. The helper lipid can be a cationic lipid. The cationic helper lipid can include one or more of DOTAP, DMTAP, DPTAP, DSTAP, DC-cholesterol, DDAB, DOGS, DODAB, DOSPA, and DOTMA. The lipid-containing particle can be liver-tropic. The lipid-containing particle can be spleen-tropic. The cholesterol or derivative thereof can be cholesterol. The PEG-based compound can be a PEG-lipid (e.g., where the PEG has a molecular weight of about 300 g/mol to about 5000 g/mol). At pH 5.5, the lipid-containing particle can have a net positive charge. The lipid-containing particle can have a diameter of about 60 nM to about 160 nM. The lipid-containing particle can have a zeta potential at pH 7.4 of about −11 mV to about −2 mV. The lipid-containing particle can have a pKa of about 5.3 to about 7.5.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1B: A library of branched and linear tail ionizable lipidoids was synthesized. To generate acrylate tails, alcohols with lipid tails totaling 10 carbons were esterified (FIG. 1A, top). Ionizable lipidoids were synthesized via Michael addition of the acrylate tails and the alkyl-amine 306(FIG. 1A, middle). The tail nomenclature denotes the number of carbons in the branched tail, the position of the branch point, and the length of the shorter branch, and the “O” denotes an ester linker between the lipid tail and amine-containing head group (FIG. 1A, bottom). FIG. 1B shows the final library, which contained lipid tails of 10 carbons with varying branch points and tail lipid lengths. Linear tail lipids with lengths of 10, 9, 8, and 6 were included for comparison.

FIGS. 2A-2G show that branched tail lipid nanoparticles had similar characteristics. LNPs were formulated with PEG C14-2000, DOPE, cholesterol, and the ionizable lipid (FIG. 2A). The LNPs were then characterized for hemolytic ability (FIG. 2B), RNA entrapment (FIG. 2C) via the RiboGreen™ Assay, LNP concentration (FIG. 2D) and size (FIG. 2E) via nanoparticle tracking analysis, zeta potential (FIG. 2F) using a ZetaSizer, and pKa (FIG. 2G) using the TNS assay. N=3-8; error bars represent SD.

FIGS. 3A-3C show that branched tail LNPs were highly potent compared to linear tail LNPs. LNPs formulated with mLuc were delivered to HepG2 cells at an RNA dose of 100 ng per 96-well (n=4), and relative luminescence units (RLU) were measured (FIG. 3A). The LNPs also were intravenously delivered to mice at a luciferase mRNA dose of 0.5 mg/kg. Three hours later, harvested organs were imaged by IVIS to quantify luminescence (FIG. 3B). The branched tail LNPs were uniformly highly potent, with most protein expression occurring in the liver and, to a lesser degree, in the spleen (FIG. 3C). Most branched-tail LNPs performed as well as 10(8)1, indicated by the dashed vertical line. Error bars represent SD. One-way ANOVA *P<0.05, ** P<0.001, *** P<0.0001, N=3.

FIGS. 4A-4G show that branched tail lipid nanoparticles are a versatile delivery vehicle. Two novel branched tail LNPs (306O₁₀(4)3 and 306O₁₀(5)1) were further examined for efficacy and compared to the benchmark LNP, 10(8)1. All panels of FIGS. 4A-4G refer to IV-delivered nanoparticles, except for the last two. Firefly luciferase mRNA was used in all panels. Both of the novel LNPs resulted in dose-dependent firefly luciferase expression (FIG. 4A). All LNPs retained efficacy following four repeat-injections over 60 days (FIG. 4B). As shown in FIG. 4C, All LNPs effectively delivered three multiplexed mRNAs: Luciferase (circles), GFP (squares), and mCherry (diamonds). The multiplexed mRNAs were delivered at a total RNA concentration of 1 mg/kg, divided equally between mRNAs. Delivery efficacy for each treatment was statistically different than untreated groups (indicated by the dashed lines). There was no difference in delivery efficacy between the tested LNPs. Branched tail LNPs that simultaneously delivered Factor VII (FVII) siRNA and luciferase mRNA (FIG. 4D) and potently delivered mRNA across multiple injection routes (FIG. 4E). All LNPs exhibited similar organ tropism following intraperitoneal (IP; FIG. 4F) and intramuscular (IM; FIG. 4G) delivery. One-way ANOVA *=P<0.05, ***=P<0.0001, ****=P<0.0001, N=3-4, Error Bars=SD.

FIG. 5 is a series of graphs plotting pKa values for each lipid nanoparticle, determined using the TNS Assay. LNPs were made at 0.05 mg/ml, and TNS fluorescence was measured as a function of pH. Data were normalized to the lowest pH measured and interpolated using GraphPad prism setting: sigmoidal, 4PL, X represents the log (concentration). The value of the pKa was determined by the LogIC50 value. Error bars=SD, n=3-8.

FIGS. 6A-6C show that branched tail lipid nanoparticle efficacy correlated with TNS values at pH 5.5 and pKa value. Lipid nanoparticles were delivered at 0.5 mg/kg of luciferase mRNA, and luminescence was measured three hours later. Total in vivo efficacy was plotted against in vitro efficacy from delivering 100 ng to HepG2 cells, but no correlation was found (FIG. 6A). However, significant correlations were found when comparing total in vivo efficacy with TNS at pH 5.5(FIG. 6B) and the surface pKa of the lipid nanoparticles (FIG. 6C). Person Correlation, P>0.05. Error bars=SD, n=3-8.

FIG. 7 shows that branched tail lipid nanoparticles overwhelmingly outperformed their linear tail counterparts. Lipid nanoparticles were delivered IV to mice at 0.5 mg/kg of luciferase mRNA, and luminescence was measured three hours later. The average total luminescent flux was compared between linear and branched tail lipid nanoparticles via a fold change. As the linear carbon tail length decreased, an increase in fold change in branched tail lipid nanoparticles efficacy was observed. Each dot represents a branched tail lipid nanoparticle. Dotted lines represent means.

FIG. 8 shows 8 ionizable lipids that were generated and used to synthesize a small library of branched tail ionizable lipidoids with 11 carbons.

FIGS. 9A-9D show that branched tail lipid nanoparticles with 11 carbons entrapped mRNA at >70%. Branched tail lipid nanoparticles with 11 carbons were formulated using PEG C-14 2000 MW, DOPE, cholesterol, and the ionizable lipid. Entrapment of luciferase mRNA was measured (FIG. 9A). Branched tail lipid nanoparticle size was determined by dynamic light scattering (FIG. 9B). Surface charge was measured by zeta potential at pH 7.4(FIG. 9C). The pKa of the lipid nanoparticles also was assessed (FIG. 9D). N=3, error cars=standard deviation.

FIGS. 10A-10B show that two potent ionizable lipids with 11 carbon branched chains were identified from in vivo screening. Branched tail lipid nanoparticles were formulated with mRNA for firefly luciferase and delivered intravenously to C57BL/6 mice at 0.5 mg/kg. Two of these were significantly more efficacious than the linear 11-carbon-tailed ionizable lipid (FIG. 10A). Organ tropism for the most potent branched tail lipid nanoparticles showed a shift to the spleen (FIG. 10B). One-way ANOVA, ** P<0.01, *** P<0.001, **** P<0.0001, N=3, error bars=standard deviation.

FIGS. 11A-11D show that top-performing branch tail lipid nanoparticles effectively delivered three RNAs. Branch tail lipid nanoparticles 11(7)2 and 11(9)1 were formulated with two mRNAs (mLuc and mCre) and an siRNA for FVII as depicted in FIG. 11A. The lipid nanoparticles were intravenously delivered to Ai9 mice at a total RNA concentration of 0.33 mg/kg for each mRNA and 0.006 mg/kg siFVII. There was no significant difference in signal between 11(7)2 and 11(9)1 for mLuc (FIG. 11B), mCre (FIG. 11C), or siFVII (FIG. 11D). One-way ANOVA, **** P<0.0001, N=3, error bars=standard deviation.

FIG. 12 includes graphs showing normalized TNS curves for calculation of pKa for the indicated lipid nanoparticles. Lipid nanoparticles were prepared at 0.05 mg/mL and TNS fluorescence was measured. Data were normalized to the lowest pH measured and interpolated using GraphPad prism setting: sigmoidal, 4PL, X is log (concentration). The value of the pKa was determined by the LogIC50 value. Error=SD, n=3-8.

FIGS. 13A-13B show that the pKa of branched tail (BRAT) lipid nanoparticles correlated with in vivo efficacy. Lipid nanoparticles were prepared at 0.05 mg/mL and TNS fluorescence was measured. Data were normalized to the lowest pH, and then the pKa was calculated. The normalized TNS fluorescence at pH 5.5, endosomal pH, had a significantly non-zero slope (FIG. 13A), but the person correlation was not significant (P=0.0887). The pKa of the BRAT lipid nanoparticle also had a significantly non-zero slope (FIG. 13B), and the person correlation coefficient had an R2 of 0.7078 and P=0.0088. N=3.

### DETAILED DESCRIPTION

Provided herein are ionizable lipids, lipid particles containing the ionizable lipids, and methods for using the ionizable lipid-containing particles for delivery of one or more agents (e.g., nucleic acids, such as one or more mRNA molecules) to organisms such as mammals, insects, and/or plants.

Lipid-containing particles are small particles or structures that include lipids and/or lipid-like materials (e.g., lipidoids). Lipid-containing particles are found in various biological forms, such as LNPs (which can be used in drug delivery systems), lipoproteins (which can transport lipids in the bloodstream), lipid droplets (which are intracellular storage organelles), exosomes (which are involved in cell-to-cell communication), vesicles (which are small membrane-bound sacs within cells), and others. In general, the lipid-containing particles (e.g., LNPs) provided herein include a mixture of: (i) an ionizable lipid; (ii) a membrane stabilizing compound (e.g., a sterol such as cholesterol, a cholesterol analogue, or a cholesterol derivative); (iii) a helper lipid; and (iv) a PEG-lipid or PEG-cholesterol conjugate.

Any appropriate ionizable lipid (or lipidoid) can be included in the lipid-containing particles (e.g., LNPs) provided herein. Suitable lipids include, for example, fats, waxes, sterols, fat-soluble vitamins, and other similar substances. Lipidoids are a class of lipid-like materials often used in biotechnology and nanomedicine, particularly for the delivery of nucleic acids such as RNA and DNA. In general, the ionizable lipids provided herein have an amine-containing head and one or more acrylate tails. Examples of suitable amine-containing heads include, without limitation, N¹-(3-aminopropyl)-N¹-methylpropane-1,3-diamine, N¹,N¹′-(propane-1,3-diyl)bis(N¹-methylethane-1,2-diamine), N¹,N¹′-(propane-1,3-diyl) bis(N¹-ethylethane-1,2-diamine), N¹,N¹′-(ethane-1,2-diyl)bis(N¹-methylethane-1,2-diamine), 4-(2-aminopropan-2-yl)-1-methylcyclohexan-1-amine, 2-(piperazin-1-yl) ethan-1-amine, and N¹-((4-)2-aminoethyl) piperazin-1-yl)methyl) ethane-1,2-diamine.

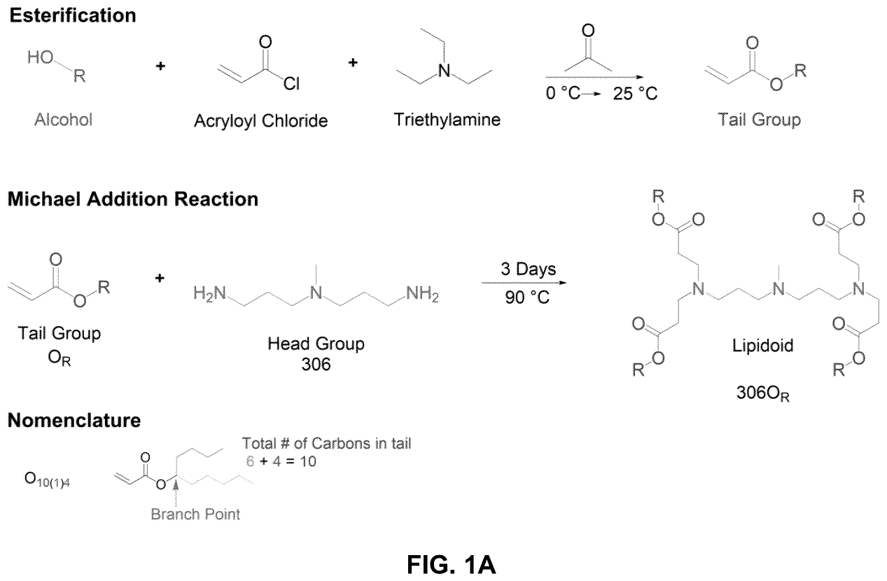

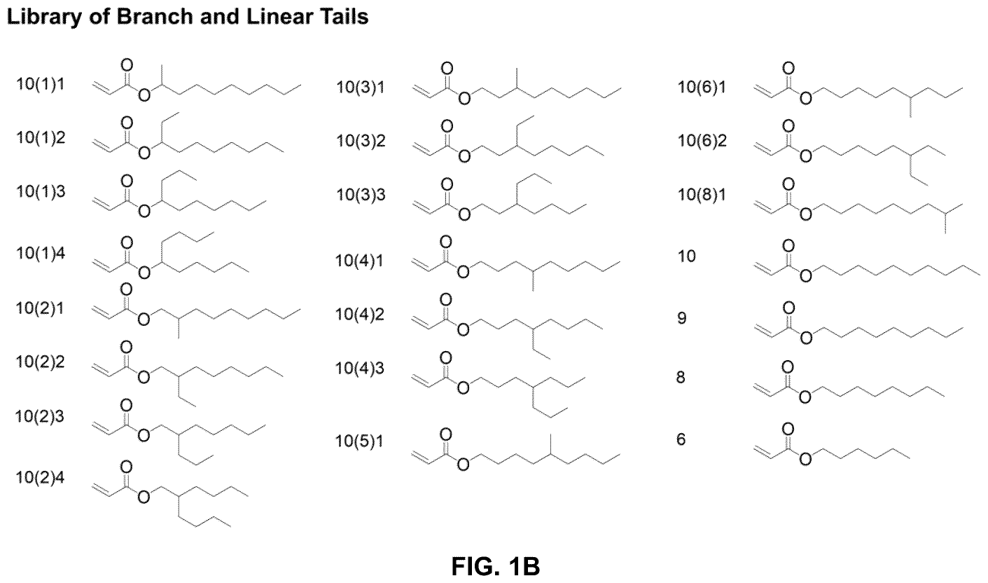

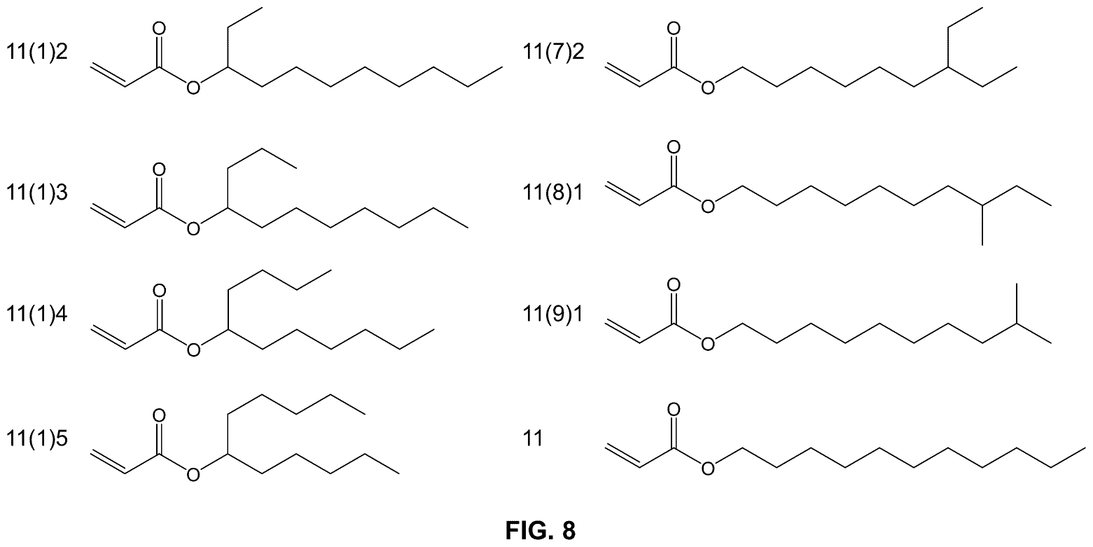

The ionizable lipids provided herein can have any appropriate number of acrylate tails. For example, an ionizable lipid provided herein can have one, two, three, four, or more than four (e.g., five, six, seven, or eight) acrylate tails. In some cases, when an ionizable lipid provided herein has more than one acrylate tail, all of the acrylate tails can be the same. In some cases, when an ionizable lipid provided herein has more than one acrylate tails, the ionizable lipid can include two or more (e.g., two, three, four, five, or more than five) different acrylate tails. Non-limiting examples of suitable acrylate tails are depicted in FIG. 1B and FIG. 8. Each acrylate tail can include a branched alkyl chain of 10 or 11 carbon atoms, such that the acrylate tail has main chain and a first branch, where the first branch includes any appropriate number of carbons. In some cases, the first branch can be one, two, three, or four carbon atoms in length. The branch can be at any appropriate position on the main alkyl chain of the acrylate tail. For example, the branch can be at the first, second, third, fourth, fifth, or sixth carbon of the main alkyl chain. In some cases, the branch can be at the first, third, fourth, fifth, or sixth carbon of the main alkyl chain. In some case, the branch is not at the second carbon of the main alkyl chain. Examples of acrylate tails that can be included in the ionizable lipids provided herein include a decan-2-yl acrylate tail (10(1)1), a decan-3-yl acrylate tail (10(1)2), a decan-4-yl acrylate tail (10(1)3), a decan-5-yl acrylate tail, (10(1)4), a 2-methylnonyl acrylate tail (10(2)1), a 2-ethyloctyl acrylate tail (10(2)2), a 2-propylheptyl acrylate tail (10(2)3), a 2-butylhexyl acrylate tail (10(2)4), a 3-methylnonyl acrylate tail (10(3)1), a 3-ethyloctyl acrylate tail (10(3)2), a 3-propylheptyl acrylate tail (10(3)3), a 4-methylnonyl acrylate tail (10(4)1), a 4-ethyloctyl acrylate tail (10(4)2), a 4-propylheptyl acrylate tail (10(4)3), a 5-methynonyl acrylate tail (10(5)1), a 6-methylnonyl acrylate tail (10(6)1), a 6-ethyloctyl acrylate tail (10(6)2), an undecan-3-yl acrylate tail (11(1)2), an undecan-4-yl acrylate tail (11(1)3), an undecan-5-yl acrylate tail (11(1)4), an undecan-6-yl acrylate tail (11(1)5), a 7-ethylnonyl acrylate tail (11(7)2), an 8-methyldecyl acrylate tail (11(8)1), or a 9-methyldecyl acrylate tail (11(9)1). The “X (Y) Z” nomenclature of the tails denotes the number of carbons in the branched tail (“X”), the position of the branch point (“Y”), and the length of the shorter branch (“Z”).

In some cases, a branched acrylate tail in an ionizable lipid provided herein can have symmetrical branches. Examples of such branched acrylate tails include 2-butylhexyl (10(2)4) acrylate tails, 4-propylheptyl (10(4)3) acrylate tails, 6-ethyloctyl (10(6)2) acrylate tails, undecan-6-yl (11(1)5) acrylate tails, 7-ethylnonyl (11(7)2) acrylate tails, and 9-methyldecyl (11(9)1) acrylate tails. Without being bound by a particular mechanism of action, such symmetrically branched acrylate tails may provide a structure that allows for enhanced ionization at the endosomal stage and/or facilitates endosomal escape. At reduced pH, lipid-containing particles can take on net positive charge, which destabilizes the endosomal membrane and causes release of the contents (e.g., RNA, in the case of lipid-containing particles carrying RNA cargo). For lipid-containing particles containing ionizable lipids having branched tails, including symmetrically branched tails, ion pairing may be stronger, which may help the endosomal escape process.

The ionizable lipids provided herein can be generated using any appropriate method. For example, an amine-containing head can be combined with one or more acrylate tails in any appropriate ratio (e.g., a stoichiometric amine-containing head: acrylate tail ratio of about 1:2 to about 1:10, such as about 1:2, about 1:3, about 1:4, about 1:5, about 1:6, about 1:7, about 1:8, about 1:9, or about 1:10), and can be mixed together at any appropriate temperature and for any appropriate length of time. For example, an amine-containing head and one or more acrylate tails can be mixed at about 60° C. to about 120° C. (e.g., about 60° C., about 70° C., about 80° C., about 90° C., about 100° C., about 110° C., or about 120° C.) for about 12 hours to about 7 days (e.g., about 12 to 24 hours, about 1 to 2 days, about 2 to 3 days, about 3 to 4 days, about 4 to 5 days, about 5 to 6 days, or about 6 to 7 days). As depicted in FIG. 1A, such a procedure can generate an ionizable lipid provided herein via a combinatorial Michael addition reaction.

Any appropriate membrane stabilizing compound (e.g., a sterol such as cholesterol, a cholesterol analogue, or a cholesterol derivative) can be included in the lipid-containing particles (e.g., LNPs) provided herein. Examples of suitable cholesterol derivatives (or analogues) include, without limitation, oxidized cholesterol, desmosterol, 7-dehydrocholesterol, ergosterol, lanosterol, ketosterone, cholesterol sulfate, dehydroergosterol, cholestratrienol, 5-cholestene, and pregnenolone. In some cases, the lipid-containing particles (e.g., LNPs) provided herein can contain cholesterol.

Any appropriate helper lipid can be included in the lipid-containing particles (e.g., LNPs) provided herein. Helper lipids can be cationic, anionic, neutral, or zwitterionic amphiphilic lipids, and along with cholesterol or a derivative thereof (e.g., a cholesterol analog), can aid in the molecular packing and stability of a lipid-containing particle (e.g., a LNP). Helper lipids also can enhance lipid nanoparticle efficacy by promoting fusion with both cell and endosomal membranes, facilitating cell uptake and endosomal release. In some cases, a lipid-containing particle provided herein can include one or more zwitterionic helper lipids. Non-limiting examples of suitable zwitterionic helper lipids include 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-distearoyl-sn-glycero-3-phosphoethanolamine (DSPE), 1,2-dipalmitoyl-sn-glycero-3-phosphocholine (DPPC), 1-palmitoyl-2-oleoyl-sn-glycero-3-phosphocholine (POPC), 1,2-dimyristoyl-sn-glycero-3-phosphocholine (DMPC), 1,2-dipalmitoyl-sn-glycero-3-phosphoethanolamine (DPPE), 1-palmitoyl-2-oleoyl-sn-glycero-3-phosphoethanolamine (POPE), 1,2-di-myristoyl-sn-glycero-3-phosphoethanolamine (DMPE), palmitoyl sphingomyelin (PSM), and sterol sphingomyelin (SSM). In some cases, a lipid-containing particle provided herein can include one or more neutral helper lipids. Non-limiting examples of suitable neutral helper lipids include triglycerides, triacylglycerols, diglycerides, diacylglycerols, and ceramides. In some cases, a lipid-containing particle provided herein can include one or more cationic helper lipids. Non-limiting examples of suitable cationic helper lipids include 1,2-dileoyl-3-trimethylammonium-propane (DOTAP), 1,2-dimyristoyl-sn-glycero-3-trimethylammonium-propane (DMTAP), 1,2 dipalmitoyl-sn-glycero-3-trimethylammoniumpropane (DPTAP), 1,2-distearoyl-sn-glycero-3-trimethylammoniumpropane (DSTAP), 3β-[N-(N′,N′-dimethylaminoethane)-carbamoyl]cholesterol (DC-cholesterol), didodecyldimethylammonium bromide (DDAB), dioctadecyloxy-propyl-glycerol (DOGS), dimethyldioctadecylammonium bromide (DODAB), dioleyloxy-propyl-trimethylammonium (DOSPA), and N-[1-(2,3-dioleoyloxy) propyl]-N,N,N-trimethylammonium chloride (DOTMA).

Any appropriate PEG-based compound can be included in the lipid-containing particles (e.g., LNPs) provided herein. PEG is a polyether compound derived from petroleum. PEG and PEG-based compounds can be used for various applications, such as drug delivery agents, solvents, adhesives, adsorbents, and tissue engineering scaffolds. PEG-based compounds that can be used in the lipid-containing particles provided herein include, without limitation, PEG-lipids (PEGylated lipids) and PEG-cholesterols (PEGylated cholesterols). PEG-lipids include a PEG moiety attached to one or more lipid moieties (e.g., a ceramide, succinoyl, or carbamate moiety). PEG-cholesterols include a PEG moiety attached to one or more cholesterol moieties. PEG-lipids and/or PEG-cholesterols can form a protective, non-aggregating, non-immunogenic shell around the surface of LNPs. Depending on the ultimate delivery route of the LNPs, the lipid group may be varied (e.g., in length) to dictate how long the PEG-lipid will be associated with the LNP, with longer lipid chains tending to remain associated with the LNP for longer time periods, and shorter lipid chains typically being useful for providing “diffusible” PEG lipids that diffuse from the lipid nanoparticle quickly to produce an LNP with increased transfection rates. The PEG moiety of a PEG-lipid or PEG-cholesterol can have a molecular weight ranging from about 300 g/mol to about 5000 g/mol (e.g., about 300 g/mol to about 500 g/ml, about 500 g/mol to about 1000 g/mol, about 1000 g/mol to about 2000 g/mol, about 1500 g/mol to about 2500 g/mol, about 2000 g/mol to about 3000 g/mol, about 2500 g/mol to about 3500 g/mol, about 3000 g/mol to about 4000 g/mol, about 3500 g/mol to about 4500 g/mol, about 4000 g/mol to about 5000 g/mol, about 1000 g/mol, about 2000 g/mol, about 3000 g/mol, about 4000 g/mol, or about 5000 g/mol). For example, the PEG moiety of a PEG-lipid or PEG-cholesterol can have a molecular weight of about 2000, which is referred to as PEG 2000. Non-limiting examples of suitable PEG-lipids include 1,2-dimyristoyl-sn-glycero-3-phosphoethanolamine-N [methoxy (polyethylene glycol)-2000], N-octanoyl-sphingosine-1-{succinyl [methoxy (polyethylene glycol)2000]}, N-palmitoyl-sphingosine-1-{succinyl [methoxy (polyethylene glycol)5000]}, 1,2-dipalmitoyl-sn-glycero-3-phosphoethanolamine-N-[methoxy (polyethylene glycol)-3000](ammonium salt), 1,2-dimyristoyl-sn-glycero-3-phosphoethanolamine-N-[methoxy (polyethylene glycol)-1000](ammonium salt), and PEG-cholesterol, such as cholesterol-(polyethylene glycol-600). In some cases, the lipid-containing particles (e.g., LNPs) in the compositions provided herein can contain a PEG-lipid. In some cases, the PEG-lipid or PEG-cholesterol can be modified with a targeting moiety, such as N-acetylgalactosamine (GalNAc) for liver targeting, or with another ligand or binding reagent, such as a polypeptide (e.g., an antibody or antibody fragment), an apolipoprotein (e.g., ApoE), a peptide, a 1,2-dimyristoyl-rac-glycero-3-methoxy (DMG) group, and/or a small molecule ligand similar to GalNAc. In some cases, a PEG alternative or a modified PEG can be used in a PEG-based compound. A non-limiting example of a PEG alternative is polysarcosine (pSAR).

The lipid containing particles provided herein can have any appropriate characteristics. For example, a lipid-containing particle provided herein can have any appropriate size. In some cases, a lipid-containing particle provided herein can have a diameter of about 50 nm to about 250 nm (e.g., about 50 to about 100 nm, about 100 to about 130 nm, about 130 to about 150 nm, about 140 to about 160 nm, about 150 to about 170 nm, about 160 to about 180 nm, about 180 to about 200 nm, or about 200 to about 250 nm). A lipid-containing particle provided herein can have any appropriate surface charge. In some cases, a lipid-containing particle provided herein can have a net negative charge at neutral pH (e.g., as determined by zeta potential). For example, a lipid-containing particle provided herein can have a zeta potential of about −11 mV to about −0.01 mV (e.g., about −11 to about-9 mV, about −9 to about −7 mV, about −7 to about −5 mV, about −5 to about −3 mV, about −3 to about −1 mV, or about −1 to about −0.1 mV). In some cases, a lipid-containing particle provided herein can have no charge, such that the lipid-containing particle has a zeta potential of zero. In some cases, a lipid-containing particle provided herein can have a net positive charge (e.g., at endosomal pH of about 5.5), such that the lipid-containing particle has a zeta potential of about 0.1 to about 11 mV. A lipid-containing particle provided herein can have any appropriate pKa. For example, a lipid-containing particle provided herein can have a pKa of about 5 to about 7.5(e.g., about 5 to about 5.5, about 5.5 to about 6, about 5.75 to about 6.25, about 5.8 to about 6.2, about 5.9 to about 6.1, about 6 to about 6.2, about 6 to about 6.5, about 6.5 to about 7, about 7 to about 7.5, about 5, about 5.5, about 5.8, about 5.9, about 6, about 6.1, about 6.2, about 6.5, about 7, or about 7.5).

Lipid-containing particles (e.g., LNPs) can be effective for delivering therapeutic agents (e.g., nucleic acids and nucleic acid analogs) to cells, often in vivo (see, e.g., Kulkarni et al., Nucl Acid Ther (2018), 28(3): 146-157; Hajj and Whitehead, Nature Rev Mat (2017), 2:17056; U.S. Publication No. 20130245107; and U.S. Pat. No. 8,754,062). As used herein, a “therapeutic agent” is any compound or composition that can be delivered to a patient to achieve a desired effect, such as a beneficial, treatment, or curative effect. Therapeutic agents include, without limitation, nucleic acids, nucleic acid anal ogs, proteins, polypeptides, small molecule drugs, antibiotics, antivirals, and cell-based therapies (e.g., CAR-T cell therapies).

In some cases, the lipid-containing particles provided herein can include a therapeutic agent that is, or comprises, a nucleic acid or nucleic acid analog. As used herein, the term “nucleic acid” includes any compound and/or substance that comprises a polymer of nucleotides (also referred to as “polynucleotides” or “oligonucleotides”). Exemplary nucleic acids include, without limitation, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, 2′-amino-α-LNA having a 2′-amino functionalization) or hybrids thereof, transfer RNAs, and short hairpin RNAs (shRNAs). Naturally occurring nucleic acids generally have a deoxyribose sugar (e.g., found in deoxyribonucleic acid (DNA)) or a ribose sugar (e.g., found in ribonucleic acid (RNA)).

Nucleic acids and nucleic acid analogs have a backbone and a sequence of nucleobases. In the context of this document, the backbone monomer residues can be any suitable nucleic acid backbone monomer residues having a negative charge, such as a ribose or deoxyribose connected to another ribose or deoxyribose by a phosphodiester bond, or a backbone residue of a nucleic acid analog monomer. The backbone monomer can include both the structural “residue” component, such as the ribose in RNA, and any active groups that are modified in linking monomers together, such as the 5′ triphosphate and 3′ hydroxyl groups of a ribonucleotide, which are modified when polymerized into RNA to result in a negatively-charged phosphodiester linkage.

For example, a therapeutic agent can be a nucleic acid or nucleic acid analog having a negatively-charged backbone, including but not limited to single-stranded DNA, single-stranded RNA, double-stranded DNA, double-stranded RNA, or modified versions of any of the preceding (e.g., versions that include one or more changes to the nucleotide components), or analogs of any of the preceding (e.g., synthetic molecules that mimic the structure and function of the original). With regard to overall structure and function of the nucleic acid or nucleic acid analog, the nucleic acid or nucleic acid analog can be, without limitation: mRNA (messenger RNA), siRNA (small interfering RNA), miRNA (microRNA), gRNA (guide RNA), tRNA (transfer RNA), rRNA (ribosomal RNA), tmRNA (transfer-messenger RNA), lncRNA (long non-coding RNA), circRNA (circular RNA), antisense RNA, ncRNA (non-coding RNA), telomerase RNA, piRNA (Piwi-interacting RNA), snRNA (small nuclear RNA), snoRNA (small nucleolar RNA), scaRNAs (small Cajal body RNA), Y RNA, eRNA (enhancer RNA), shRNA (small hairpin RNA), stRNA (small temporal RNA), DNA, chloroplast DNA, cDNA (complementary DNA), gDNA (genomic DNA), Hachimoji DNA, mitochondrial DNA, msDNA (multicopy single-stranded DNA), XNA (xeno nucleic acid), glycol nucleic acid, threose nucleic acid, hexose nucleic acid, LNA (locked nucleic acid), PNA (peptide nucleic acid), morpholino oligomer, antisense oligonucleotide, ribozyme, deoxyribozyme, aptamer, cloning vector, phagemid, plasmid, lambda phage, cosmid, fosmid, or artificial chromosome.

RNA therapeutics are a class of RNA-based treatments that target specific genes or genetic pathways with high specificity. The use of RNA therapeutics can allow for transient expression or inhibition, which can reduce long-term side effects. RNA therapeutics utilize various forms of RNA to treat diseases such as, without limitation, infectious diseases, cancer, genetic disorders, cardiovascular diseases, and neurological diseases. Research has been ongoing since the 1990s, with significant success in cancer therapy in the early 2010s (see, e.g., Sahin et al., Nature Reviews Drug Discovery, 13:759-780, 2014). The RNA used in an RNA therapeutic can include, for example, mRNA, siRNA, short hairpin RNA (shRNA), microRNA (miRNA), antisense RNA, gRNA, long non-coding RNA, transfer RNA, ribosomal RNA, double-stranded RNA (dsRNA), and/or an RNA aptamer. In some cases, for example, an mRNA-based therapy can be used.

mRNA-based therapies can trigger synthesis of proteins by delivering coding mRNA into cells, making such therapies particularly useful in vaccine development (see, e.g., DeFrancesco, Nature Biotechnology, 35:193-197, 2017). The coding mRNA can be designed as a blueprint to generate a protein of interest (e.g., a reporter protein, a functional protein, or an antigen). In some embodiments, a protein of interest can be an antigen produced by a pathogen (e.g., a virus) or by a cancer cell. Such protein molecules can stimulate an adaptive immune response that teaches the body to identify and destroy the corresponding pathogen or cancer cells (see, e.g., Bae and Park, Advanced Drug Delivery Reviews, 158:4-16, 2020). mRNA vaccines (e.g., the Pfizer-BioNTech COVID-19 vaccine and the Moderna COVID-19 vaccine) were developed for use in combating the coronavirus disease during the COVID-19 pandemic (see, Noor, Current Clinical Microbiology Reports, 8(3): 178-185, 2021). In some cases, a designed mRNA can encode a reporter such as firefly luciferase. An mRNA encoding a desired protein (e.g., an mRNA encoding luciferase) can be delivered into cells using lipid-containing particles (e.g., LNPs) to produce the protein (e.g., luciferase) in vitro, through cell culture, and in vivo, such as in mouse models or in any other appropriate mammal (e.g., humans, non-human primates, rats, rabbits, cows, pigs, sheep, dogs, and/or cats). The mammal can be healthy or can have a disease, disorder, or clinical condition.

siRNA-based therapies can reduce or eliminate the expression of a particular gene. This process is referred to as gene knockdown, and is achieved by introducing synthetic siRNA molecules into cells where they can bind to and degrade the corresponding mRNA of the target gene. siRNAs are designed to be complementary to a specific mRNA sequence of a target gene (e.g., a gene involved in inflammation). Once inside the cell, the siRNA molecule can bind to the target mRNA, which causes the target mRNA to be recognized and degraded by the cell's machinery. Without the mRNA, the cell produces less (or no) protein from that gene, effectively silencing or knocking down its expression. siRNAs typically are highly specific for their target mRNA sequences, which can minimize off-target effects. Although the effect of siRNAs is temporary and generally only lasts for several days, siRNAs can be useful to treat or alleviate the symptoms of various diseases.

The lipid-containing particles provided herein can be generated using any appropriate method. For example, lipid-containing particles (e.g., LNPs) can be generated by combining an ionizable lipid or lipidoid provided herein, cholesterol or a cholesterol derivative, a helper lipid, and a PEG-based compound in any appropriate amounts or ratios. In some cases, lipid-containing particles (e.g., LNPs) can be prepared by combining:
about 25 mol % to about 70 mol % (e.g., about 25 to about 30 mol %, about 30 to about 40 mol %, about 35 to about 45 mol %, about 40 to about 50 mol %, about 45 to about 55 mol %, about 50 to about 60 mol %, about 55 to about 65 mol %, about to 60 to about 70 mol %, about 25 mol %, about 30 mol %, about 40 mol %, about 50 mol %, about 60 mol %, or about 70 mol %) of an ionizable lipid provided herein (e.g., 306O₍₁₀₍₁₎₂₎, 306O₍₁₀₍₂₎₁₎, 306O₍₁₀₍₂₎₂₎, 306O₍₁₀₍₂₎₃₎, 306O₍₁₀₍₃₎₂₎, 306O₍₁₀₍₄₎₂₎, 306O₍₁₀₍₄₎₃₎, 306O₍₁₀₍₅₎₁₎, 306O₍₁₀₍₆₎₂₎, 306O₍₁₁₍₁₎₂₎, 306O₍₁₁₍₁₎₃₎, 306O₍₁₁₍₁₎₄₎, 306O₍₁₁₍₁₎₅₎, 306O₍₁₁₍₇₎₂₎, 306O₍₁₁₍₈₎₁₎, or 306O₍₁₁₍₉₎₁₎;
about 2 mol % to about 70 mol % (e.g., about 2 to about 10 mol %, about 5 to about 15 mol %, about 10 to about 20 mol %, about 20 to about 30 mol %, about 30 to about 40 mol %, about 35 to about 45 mol %, about 40 to about 50 mol %, about 45 to about 55 mol %, about 50 to about 60 mol %, about 55 to about 65 mol %, about to 60 to about 70 mol %, about 5 mol %, about 10 mol %, about 15 mol %, about 20 mol %, about 25 mol %, about 30 mol %, about 40 mol %, about 50 mol %, about 60 mol %, or about 70 mol %) of a helper lipid (e.g., DOPE, DSPC, or DOTAP);
about 5 to about 70 mol % (e.g., about 5 to about 10 mol %, about 10 to about 20 mol %, about 20 to about 30 mol %, about 25 to about 35 mol %, about 30 to about 40 mol %, about 35 to about 45 mol %, about 40 to about 50 mol %, about 45 to about 55 mol %, about 50 to about 60 mol %, about 60 about 70 about, about 5 mol %, about 10 mol %, about 15 mol %, about 20 mol %, about 25 mol %, about 30 mol %, about 35 mol %, about 38.5 mol %, about 40 mol %, about 45 mol %, about 50 mol %, about 60 mol %, or about 70 mol %) cholesterol or a cholesterol derivative (e.g., cholesterol); and about 0.2 to about 10 mol % (e.g., about 0.2 to about 1 mol %, about 0.5 to about 1.5 mol %, about 1 to about 2 mol %, about 1.5 to about 2.5 mol %, about 2 to about 3 mol %, about 3 to about 5 mol %, about 5 to about 7.5 mol %, about 7.5 to about 10 mol %, about 0.5 mol %, about 1 mol %, about 1.5 mol %, about 2 mol %, about 2.5 mol %, about 3 mol %, about 4 mol %, about 5 mol %, about 6 mol %, about 7 mol %, about 8 mol %, about 9 mol %, or about 10 mol %) of a PEG-based compound (e.g., PEG or a PEG-lipid).

In some cases, lipid-containing particles can be prepared by combining the above components at a molar ratio of about 35:46.5:16:2.5(lipidoid: cholesterol: helper lipid: PEG).

Lipid-containing particles (e.g., LNPs) prepared as described herein can be combined with any appropriate cargo (e.g., a nucleic acid or other therapeutic agent, and/or a marker). In some cases, the concentration of RNA as a cargo in a lipid-containing particle (e.g., for in vitro cell culture) can be from about 0.001 mg/mL to about 2 mg/mL (e.g., from about 0.001 mg/mL to about 1.5 mg/mL, from about 0.003 mg/mL to about 1.5 mg/mL, from about 0.005 mg/mL to about 1.5 mg/mL, from about 0.003 mg/mL to about 1 mg/mL, from about 0.003 mg/mL to about 0.5 mg/mL, from about 0.005 to about 1.5 mg/mL, from about 0.005 to about 1 mg/mL, from about 0.005 to about 0.5 mg/mL, about 0.001 mg/mL, about 0.003 mg/mL, about 0.005 mg/mL, about 0.01 mg/mL, about 0.03 mg/mL, about 0.05 mg/mL, about 0.08 mg/mL, about 0.1 mg/mL, about 0.2 mg/mL, about 0.3 mg/mL, about 0.4 mg/mL, about 0.5 mg/mL, about 1 mg/mL, about 1.5 mg/mL, or about 2 mg/mL). In some cases, the concentration of mRNA as a cargo in a lipid-containing particle (e.g., for in vivo use) can be from about 0.01 mg/mL to about 10 mg/mL (e.g., from about 0.01 mg/mL to about 1 mg/mL, from about 0.03 mg/mL to about 3 mg/mL, from about 0.05 mg/mL to about 5 mg/mL, from about 0.1 mg/mL to about 2 mg/mL, from about 0.3 mg/mL to about 3 mg/mL, from about 0.5 mg/mL to about 5 mg/mL, from about 1 mg/mL to about 3 mg/mL, from about 3 to about 5 mg/mL, about 0.01 mg/mL, about 0.03 mg/mL, about 0.05 mg/mL, about 0.1 mg/mL, about 0.3 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL). In some cases, when a lipid-containing particle includes mRNA, the ratio of lipidoid: mRNA (weight/weight) in the lipid-containing particle can be from about 5:1 to about 30:1(e.g., about 5:1, about 8:1, about 10:1, about 12:1, about 15:1, about 20:1, about 25:1, or about 30:1).

This document also provides compositions that include lipid-containing particles (e.g., LNPs) with lipidoid components as provided herein, in combination with a pharmaceutically acceptable carrier. Pharmaceutically acceptable carriers include, for example, pharmaceutically acceptable solvents, suspending agents, or any other pharmacologically inert vehicles for delivering therapeutic agents to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Examples of suitable pharmaceutically acceptable carriers include, without limitation: water; saline solution; binding agents (e.g., polyvinylpyrrolidone or hydroxypropyl methylcellulose); fillers (e.g., lactose or dextrose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate). In some cases, a composition provided herein can include one or more sugars (e.g., sucrose and/or trehalose) that can act as LNP stabilization/preservation agents.

Any appropriate method can be used to determine the effectiveness of a lipid-containing particle (e.g., LNP) provided herein. For example, any appropriate method can be used to determine the effectiveness of a lipid-containing particle (e.g., a LNP) to deliver a cargo (e.g., a therapeutic agent or a nucleic acid encoding a detectable marker) to a cell in vitro or in vivo. For example, in vitro efficacy can be evaluated by contacting cells in culture (e.g., primary cells obtained from a mammal, or cells from a cell line) with LNPs formulated with nucleic acid (e.g., mRNA) encoding a marker whose expression can be detected (e.g., luciferase or green fluorescent protein (GFP)). After incubating the LNPs with the cells, the cells can be assessed by measuring fluorescence or luminescence, for example, to determine whether (and the extent to which) the marker has been expressed in the cells. In some cases, in vivo efficacy can be assessed by administering LNPs formulated with nucleic acid (e.g., mRNA) encoding a marker whose expression can be detected (e.g., luciferase or GFP) to a mammal (e.g., a mouse or a rat) or a zebrafish, and after an appropriate length of time (e.g., 12 hours to 3 days after administration) assessing one or more tissues and/or organs (e.g., liver, spleen, lungs, heart, pancreas, kidneys, brain, muscle, and/or intestine) from the mammal for expression of the marker by, for example, measuring fluorescence or luminescence of the tissue(s) or organ(s). In some cases, a mammal can be administered LNPs formulated with nucleic acid (e.g., mRNA) encoding a marker whose expression can be detected (e.g., luciferase or GFP), and after an appropriate length of time, one or more tissues and/or organs from the mammal can be harvested for flow cytometry analysis to determine whether particular types of cells within the tissue(s) and/or organ(s) contain the marker.

In some cases, the lipid-containing particles provided herein can have specificity (also referred to as “tropism” or “targeting”) for one or more particular organs, tissues, or types of cells within an organ or tissue. As used herein, the terms “specificity,” “tropic” or “tropism,” and “targeting,” with regard to a particular lipid-containing particle (e.g., a LNP containing a lipidoid provided herein), mean that the lipid-containing particle is more likely to interact with (e.g., deliver a cargo to) the organ(s), tissue(s), and/or cell type(s) for which it has specificity, and is less likely to interact with other organs, tissues, and/or cell types. In some cases, a lipid-containing particle can have a preference for certain cell types within an organ or tissue. As described in the Examples herein, for example, lipid-containing particles provided herein can target the spleen and/or the liver. In some cases, when a lipid-containing particle is said to be “spleen-tropic,” at least 15% (e.g., at least 20%, at least 25%, or at least 30%) of a detectable signal derived from the lipid-containing particle is found in the spleen or spleen tissue, with the remaining percentage of the detectable signal being found in other tissues or organs. In some cases, when a lipid-containing particle is said to be “liver-tropic,” at least 15% (e.g., at least 20%, at least 25%, or at least 30%) of a detectable signal derived from the lipid-containing particle is found in the liver or liver tissue, with the remaining percentage of the detectable signal being found in other tissues or organs. In some cases, inclusion of an ionizable lipid provided herein in a lipid-containing particle can lead to an increase in spleen tropism, as compared to an ionizable lipid having a comparable number of carbons but lacking a branch. As described in Example 2 herein, for example, lipid-containing particles that included 306O(11(7)2) or 306O(11(9)1) had increased spleen tropism as compared to lipid-containing particles containing 306O₁₁.

Methods that include delivering RNA into cells can be useful in research and therapeutic applications, including gene silencing, gene editing, and mRNA-based therapeutics. As described herein, RNA delivery can be achieved via LNPs, which can avoid issues encountered with delivery of naked, single-stranded RNA (which is prone to nuclease degradation, can activate the immune system, and is too large and negatively charged to passively cross the cell membrane). Thus, this document provides methods that include delivering, to a mammal in need thereof, a lipid-containing particle (e.g., a LNP) described herein containing one or more therapeutic agents. In some cases, the one or more therapeutic agents can include nucleic acid (e.g., mRNA). The methods can include administering to a mammal a lipid-containing particle (e.g., LNP) that encapsulates the therapeutic agent (e.g., mRNA).

The methods provided herein can be used for delivering an agent (e.g., a therapeutic agent) to an organism (e.g., a mammal such as a human, non-human primate, mouse, rat, rabbit, dog, cat, horse, cow, pig, or sheep, an insect, or a plant). The methods can include administering to the subject a lipid-containing particle (e.g., a LNP) containing the agent. The lipid-containing particle (e.g., LNP) can be administered to the subject via any appropriate route. For example, a lipid-containing particle (e.g., a LNP) can be administered intravenously, intramuscularly, subcutaneously, intraocularly, intratumorally, orally, intrathecally, and/or intradermally.

As described herein, in some cases, the agent (e.g., the therapeutic agent) contained within the lipid-containing particle (e.g., LNP) used in the methods provided herein can be a nucleic acid (e.g., an RNA). The RNA can be a mRNA, siRNA, shRNA, miRNA, antisense RNA, guide RNA, long non-coding RNA, transfer RNA, ribosomal RNA, dsRNA, RNA aptamer, or any combination thereof. In some cases, an RNA encoding a marker (e.g., an mRNA encoding a luciferase polypeptide or a fluorescent polypeptide such as GFP, a yellow fluorescent polypeptide, or a red fluorescent polypeptide) can be used in the methods described herein. Luciferase is an enzyme that catalyzes a bioluminescent reaction, producing light as a byproduct. This reaction can be utilized in various biological and medical research applications, particularly in reporter assays to study gene expression and cellular processes. The bioluminescence produced by luciferase encoded by the mRNA encapsulated in the lipid-containing particle (e.g., LNP), or the fluorescence produced by a fluorescent marker encoded by the mRNA encapsulated in the lipid-containing particle (e.g., LNP) can be used to assess the efficacy of mRNA delivery.

In some cases, a lipid-containing particle provided herein can contain more than one (e.g., two, three, four, or more than four) RNA molecule (also referred to as a multiplex RNA formulation). For example, a lipid-containing particle provided herein may contain two different mRNA molecules, three different mRNA molecules, two different mRNA molecules and one or more (e.g., one, two, three, or more) siRNA molecules, or three different RNA molecules and one or more siRNA molecules. When two or more mRNA molecules are included in a lipid-containing particle provided herein, the mRNA molecules may encode polypeptides that have separate activities, or the mRNA molecules may encode polypeptides that function together. For example, a lipid-containing particle can contain an mRNA encoding the light chain of an antibody as well as an mRNA encoding the heavy chain of the antibody. When an siRNA is included in a lipid-containing particle provided herein, the siRNA can be targeted to any appropriate mRNA within the organism to which the lipid-containing particle is to be delivered. In some cases, an siRNA can be targeted to an mRNA encoding an inflammatory protein (e.g., tumor necrosis factor alpha (TNF-α), interleukin-1 beta (IL-1β), IL-6, IL-8, or interferon-gamma (IFN-γ)). In such cases, the siRNA can reduce inflammation in the organism.

When a lipid-containing particle (e.g., a LNP) provided herein includes one or more RNA molecules, the lipid-containing particle can be administered to an organism (e.g., a mammal) at an RNA (e.g., mRNA) dose of about 0.01 to about 10 mg/kg. For example, a lipid-containing particle (e.g., a LNP) can be administered at an RNA (e.g., mRNA) dose of about 0.01 to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.5 mg/kg, about 0.5 mg/kg to about 1 mg/kg, about 1 mg/kg to about 2 mg/kg, about 2 mg/kg to about 5 mg/kg, about 5 mg/kg to about 8 mg/kg, or about 8 mg/kg to about 10 mg/kg, or at a dose of about 0.01, about 0.05, about 0.1, about 0.2, about 0.3, about 0.4, about 0.5, about 0.6, about 0.7, about 0.8, about 0.9, about 1, about 1.5, about 2, about 2.5, about 3, about 4, about 5, about 6, about 7, about 7.5, about 8, about 9, or about 10 mg/kg). In some cases, a lipid-containing particle provided herein (e.g., a LNP) can be administered at an RNA (e.g., mRNA) dose of about 0.5 mg/kg.

In some cases, the methods described herein can be used to treat a clinical disorder in a subject (e.g., mammal). As used in this context, to “treat” means to ameliorate (e.g., reduce or eliminate) at least one symptom of a disorder. Administration of a therapeutically effective amount of a lipid-containing particle described herein can result in more targeted LNP-mediate mRNA delivery. Thus, the methods described herein provide an approach to enhance the effectiveness of RNA therapeutics.

Disorders that can be treated according to the methods provided herein include, without limitation, liver disorders, immune disorders, and diseases in which one or more genes or proteins is dysregulated. Liver disorders that can be treated as described herein include, without limitation, alpha-1 antitrypsin deficiency, hereditary hemochromatosis, Wilson's disease, hereditary tyrosinemia, glycogen storage diseases, viral hepatitis, familial hypercholesterolemia, nonalcoholic fatty liver disease, primary hyperoxaluria, acute intermittent porphyria, hepatocellular carcinoma, paroxysmal nocturnal hemoglobinuria, and combinations thereof. For example, a liver disorder in a mammal can be treated by administering a lipid-containing particle provided herein that is liver-tropic (e.g., a LNP containing a lipidoid provided herein that targets cells in the liver). In some cases, the clinical disorder can be an inflammatory condition, an infectious disease, an autoimmune disease, a respiratory disease, a cancer, a genetic disorder, a metabolic disease, or any combination thereof. In some cases, a mammal having a disorder can be treated by administration of lipid-containing particles containing two or more mRNA molecules (e.g., mRNAs encoding different antibody chains or other combinations of polypeptides) and, in some cases, an siRNA (e.g., an siRNA targeted to an inflammatory polypeptide. Examples of such therapies include, without limitation, immunotherapies (e.g., for vaccination, to treat cancer, or to treat an autoimmune disorder), therapies for disorders such as COVID-19 and Rift Valley Fever Virus, and protein replacement in combination with an anti-inflammatory siRNA.

Generally, the methods provided herein can include administering a therapeutically effective amount of a lipid-containing particle (e.g., LNP) described herein to a subject that is in need thereof or has been determined to be in need of, such treatment. The lipid-containing particle (e.g., LNP) encapsulates a therapeutic agent for the treatment needed. The therapeutic agent can be mRNA, siRNA, shRNA, miRNA, antisense RNA, guide RNA, long non-coding RNA, transfer RNA, ribosomal RNA, dsRNA, or RNA aptamers.

Effective doses can vary depending on the severity of the disorder, the route of administration, the age and general health condition of the subject, excipient usage, the possibility of co-usage with other therapeutic treatments, and the judgment of the treating clinician. An effective amount of a composition containing one or more lipid-containing particles (e.g., LNPs) described herein can be any amount that reduces one or more symptoms of the disorder (e.g., by at least 10, 25, 35, 45, 50, 55, 65, 75, 80, 90, or 100 percent) within a subject (e.g., a mammal), without producing severe toxicity in the mammal. As described herein, and an effective dose of a lipid-containing particle (e.g., LNP) can be an mRNA dose of about 0.01 to about 10 mg/kg (e.g., about 0.01 to about 0.05 mg/kg, about 0.05 to about 0.1 mg/kg, about 0.1 to about 0.5 mg/kg, about 0.5 to about 1 mg/kg, about 1 to about 3 mg/kg, about 3 to about 5 mg/kg, about 5 to about 7.5 mg/kg, about 7.5 to about 10 mg/kg, about 0.05, about 0.1, about 0.3, about 0.5, about 0.75, about 1, about 2.5, about 3, about 5, or about 10 mg/kg). The effective amount can remain constant or can be adjusted as a sliding scale or variable dose depending on the mammal's response to treatment. Various factors can influence the actual effective amounts used for a particular application. For example, the frequency of administration, duration of treatment, use of multiple therapeutic agents, route of administration, severity of disorder, or risk level for development of the same or another disorder in the mammal being treated may require an increase or decrease in the actual effective amount administered.

If a particular mammal fails to respond to a particular amount of a lipid-containing particle (e.g., LNP), then the amount of the lipid-containing particle can be increased by, for example, two-fold. After receiving the higher amount, the mammal can be monitored for both responsiveness to the treatment and toxicity symptoms, and adjustments can be made accordingly. The effective amount can remain constant or can be adjusted as a sliding scale or variable dose depending on the mammal's response to treatment. Various factors can influence the actual effective amount used for a particular application. For example, the frequency of administration, duration of treatment, route of administration, and severity of the disorder may require an increase or decrease in the actual effective amount administered.

The frequency of administration of one or more lipid-containing particles (e.g., LNPs) to a subject (e.g., a mammal) can be any frequency that reduces a symptom of a disorder in the subject, without producing significant toxicity to the subject. For example, the frequency of administration of a lipid-containing particle (e.g., LNP) can be from about four times daily to about once a day, from about once daily to three times a week, from about three times a week to about twice a week, from about twice a week to about once a week, or from about once a week to about once a month (e.g., from about once a week to about once every other week or about once every three weeks). The frequency of administration can remain constant or can be variable during the duration of treatment. A course of treatment with a lipid-containing particle (e.g., LNP) described herein can include rest periods. For example, a LNP can be administered daily over a one-week period followed by a one-week rest period, and such a regimen can be repeated multiple times. As with the effective amount, various factors can influence the actual frequency of administration used for a particular application. For example, the effective amount, duration of treatment, use of multiple therapeutic agents, route of administration, and severity of the disorder may require an increase or decrease in administration frequency.

An effective duration for administering one or more lipid-containing particles (e.g., LNPs) to a subject (e.g., a mammal) can be any duration that reduces a symptom of a disorder in the mammal, without producing significant toxicity to the mammal. In some cases, the effective duration can vary from several days to several months to several years or more. Multiple factors can influence the actual effective duration used for a particular treatment. For example, an effective duration can vary with the effective amount, frequency of administration, use of multiple therapeutic agents, route of administration, and severity of the disorder being treated.

In some cases, the progression of a disorder in a subject (e.g., a mammal) or the severity of one or more symptoms related to the disorder in the subject being treated can be monitored. Any appropriate method can be used to determine whether or not a subject having a disorder is effectively being treated. In some cases, the progression of a disorder in a mammal or the severity of one or more symptoms related to the disorder in the mammal being treated can be monitored. Any appropriate method can be used to determine whether or not a mammal is effectively being treated. For example, clinical scanning techniques (e.g., computed tomography (CT), positron emission tomography (PET)/CT, bone scan, magnetic resonance imaging (MRI)), and/or measurement of one or more markers in a biological sample can be used to determine the presence or absence of a disorder such as cancer within a mammal (e.g., a human) being treated. In such cases, a reduced number of tumor cells, reduced tumor size, or a reduction in a tumor marker can indicate effective treatment.

The invention will be further described in the following examples, which do not limit the scope of the invention described in the claims.

### EXAMPLES

### Example 1—Branched Tail Lipid Nanoparticles Outperform Linear Counterparts for mRNA delivery

### METHODS AND MATERIALS

Synthesis of Lipidoid Tails: Branched tails for lipidoids were synthesized by reacting alcohols (TABLE 1) with acryloyl chloride (Alfa Aesar) and trimethylamine (Sigma Aldrich) in a molar ratio of 1:1.5:2 in reagent grade acetone (ACS Grade, Spectrum) in a round bottom flask on ice. Ice was removed after about ten minutes. The flask was allowed to equilibrate to room temperature and then reacted for two hours. Quenching of the reaction was done with 3 mL of deionized water for ten minutes. The product was rotary evaporated for about 1.5 hours and then dissolved in ethyl acetate and placed in a separation funnel. Four washes were done to remove contaminants: (1) NaCl (saturated) and water at a 1:1 molar ratio, (2)1N HCl and water at a 1:1 molar ratio, (3) NaHCO₃(saturated), and (4) NaCl (saturated). The product was then retrieved and 3 to 6 mg of 2,5 Di-tert-butylhydroquinone (Sigma Aldrich) was added to prevent polymerization. Magnesium sulfate (Fisher Chemicals) was added to remove water and then vacuum filtered out. The product was rotary evaporated to remove the solvent. The tail was then purified using flash chromatography on an ISCO CombiFlash system equipped with a silica column (solid-phase silica). The sample was loaded onto the column pre-equilibrated with dichloromethane (DCM). Elution was performed using a gradient of DCM: methanol: ammonium hydroxide (60:30:10, v/v/v) that did not exceed 50%.

Synthesis of Lipidoids: As described elsewhere, lipidoids were synthesized through the addition of alky-acrylates to amines (Whitehead et al., Nat Commun (2014), 5:4277). Amine 3,3′-Diamino-N-methyldipropylamine (306, Sigma Aldrich), was reacted with custom synthesized alky-acrylates of different lengths and branching. Linear acrylate tails were purchased from Sartomer and TCI Chemicals. Acrylates were combined with amines using a molar ratio of 4:1 at 90° C. for 3-7 days in a glass scintillation vial. Fully substituted lipids were confirmed with mass spectrometry (TABLE 2).

Formation of Lipid Nanoparticles: Lipid nanoparticles were formulated by mixing lipidoid, cholesterol (Sigma Aldrich), 1,2-dioleoyl-sn-glycero-3-phosphoethanolamine (DOPE, Avanti Polar Lipids), and C14-PEG2000(Avanti Polar Lipids) in a solution of ethanol and 10 mM sodium citrate at molar ratios of 35:46.5:16:2.5. mRNA was diluted in 10 mM sodium citrate buffer. Lipidoid and mRNA mixtures were combined and then diluted to the desired concentration using phosphate-buffered saline. Lipid nanoparticles had a final weight ratio of 1:10 mRNA to lipidoid. For in vivo experiments, all lipid nanoparticles were dialyzed against PBS pH 7.4 for at least one hour.

mRNAs: Luciferace mRNA used for screening was obtained using methods described elsewhere (Pine et al., Mol Ther (2023), 31:2702-2714). mRNA also was provided by Translate Bio (now Sanofi) and obtained from TriLink Biotechnologies.

Concentration and Size of Lipid Nanoparticles. Lipid nanoparticles were analyzed by nanoparticle tracking as described elsewhere (Yerneni et al., J Extracell Vesicles (2021), 10: e12155; and Yerneni et al., mBio (2021), 12(4): e0165721) using the NanoSight LM10 system (NanoSight, Ltd., Amesbury, UK) configured with a 405-nm laser and a high-sensitivity digital camera system (OrcaFlash2.8, Hamamatsu C11440; NanoSight, Ltd.). The camera shutter speed was fixed at 30.01 ms, and camera gain was set to 500. Videos were collected and analyzed using the NTA software (version 2.3), with the minimal expected particle size, minimum track length, and blur setting all set to automatic. LNPs were synthesized and dialyzed to a final concentration of 0.05 mg/mL as described above, and were assessed on the NTA with n=5 for each run and replicated three times. Data represent an average of three replicates.

TNS Ionization and pKa values. TNS assays were conducted as described elsewhere (Hajj et al., supra) to evaluate lipid nanoparticle ionization. Briefly, 250 AL of a buffer consisting of 150 mM sodium chloride, 20 mM sodium phosphate, 20 mM ammonium acetate, and 25 mM ammonium citrate was added to each well. Then 10 AL of 0.16 mM stock solution of 2-(p-toluidinyl) naphthalene-6-sulphonic acid (TNS, Sigma Aldrich) in DI water was added along with 5 μL of 0.05 mg/mL mRNA LNP. Fluorescence intensity was measured at an excitation of 322 nM and an emission of 431 nm (Tecan Spark). The pKa of lipid nanoparticles was calculated by performing the TNS assay at pH 2 to 12 in increments of 1. A pH of 7.4 was used instead of 7 since pH 7.4 approximates physiological blood pH, and pH 5.5 was used instead of 5 since pH 5 approximates the late-stage endosomal pH. Data were normalized to the lowest pH measured and interpolated using GraphPad prism settings: sigmoidal, 4PL, X is log (concentration).

Hemolysis Assay: Human Red blood cells (Innovative Research, Novi, MI) were diluted in PBS to make a 4% v/v RBC solution. 100 μL RBC solution was seeded in clear round bottom 96-well plates (Corning, Corning, NY), and an equal volume of blank lipid nanoparticles (containing no mRNA) formulated at 0.08, 0.06 and 0.02 mg/mL equivalent mRNA concentration was added to the solution. PBS and 1% Triton-X were used as negative and positive controls, respectively. The solution was incubated for 90 minutes at 37° C. Cells were centrifuged at 500 RCF, and 100 μL supernatant was transferred to a transparent flat bottom 96-well plate (Corning, Corning, NY). Absorbance was measured at 540/640 nm using a Synergy H1 microplate reader (BioTek Instruments, Winooski, VT).

Entrapment: To measure RNA entrapment, intact and lysed nanoparticles were measured for RNA content using a Quant-iT™ RiboGreen™ RNA Assay Kit according to the manufacturer's instructions, Lipid nanoparticles were prepared at a concentration of 0.005 mg/mL with luciferase mRNA and then diluted tenfold. Briefly, lipid nanoparticles were diluted in equal volumes of Tris-EDTA buffer or 2% Triton X-100 in DI water buffer. An equal volume of RiboGreen™ reagent was then added to each sample and incubated at 37° C. for 15 minutes. The fluorescence was read on a Tecan Spark® Multimode Microplate Reader, The excitation signal was 480 nm and the emission 520 nm.

Zeta Potential: Nanoparticle zeta potential was determined by dynamic light scattering (Malvern Zetasizer Nano ZSP). Lipid nanoparticles were diluted 1:100 from 0.005 mg/mL for testing. Three technical replicates were conducted on each sample for both size and surface zeta potential.

In Vitro Testing of Lipid Nanoparticles: HepG2 cells were maintained at 37° C. and 5% CO₂ in high glucose Dulbecco's Modified Eagles Medium (DMEM, Thermo Fisher). Fetal bovine serum at 10% by volume, 1% penicillin, and streptomycin by volume were added to DMEM. Cells were seeded in a 96-well plate at 50,000 cells per well in 180 μL of media for 24 hours before transfection. HepG2 cells were transfected at an mRNA concentration of 100 ng per well and incubated for 24 hours. After transfection, Bright-GloM Luciferase Assay System (Promega) was used to measure firefly luciferase activity.

In Vivo Testing of Lipid Nanoparticles. Female C57BL/6 mice about 6 weeks in age were used. Lipid nanoparticles were intravenously injected via the tail vein at a concentration of 0.5 mg/kg of mRNA encoding for firefly luciferase. About 3 hours after injection, 130 μL of d-luciferin (PerkinElmer) was administered intraperitoneal at a concentration of 30 mg/mL. Fifteen minutes after intraperitoneal injection, mice were sacrificed and organs were imaged using the In Vivo Imaging Instruments (IVIS).

Repeated Dosage: Lipid nanoparticles were dosed on days 0, 14, 42, and 56. Live images were taken of the mice under isoflurane. Lipid nanoparticles were intravenously injected via the tail vein at a concentration of 0.5 mg/kg of mRNA encoding firefly luciferase. Three hours after injection, 130 AL of d-luciferin (PerkinElmer) was administered intraperitoneally at a concentration of 30 mg/mL. Fifteen minutes later, mice were sacrificed and organs were imaged using the IVIS.

Factor VII Assay: Factor VII levels were measured using the Hyphen Biomed BIOPHEN™ FVII assay. For the standard and calibration curve, 5 mouse samples were pooled and then diluted. Samples were diluted at 1:1500 and assessed per the manufacturer's protocol. Absorbance was measured at 540/640 nm on a Synergy HI microplate reader.

Statistical Analysis. Living Image software was used to analyze data. All statistical analysis was conducted using GraphPad Prism (La Jolla, CA). Standard deviation is abbreviated as “SD” in figure captions. Significance was determined by Tukey's multiple comparisons test.

### RESULTS

### Synthesis of Branched Tail Ionizable Lipids

A library of branched tail ionizable lipids was synthesized that contained ester linkers, branching, and a hydrophobic chain of 10 carbons. To synthesize the branched tail structures, alcohols were converted to acrylates (FIG. 1A, top). Purified branched tails were then combined with amine-containing head group 306 via Michael addition to obtain the final branched tail ionizable lipid (FIG. 1B, middle). Amine-containing head group 306 was selected given its high success rate for producing efficacious lipid nanoparticles (Whitehead et al., supra; and Hajj et al., supra). The ionizable lipids were confirmed to be fully substituted and four-tailed via mass spectrometry (TABLE 1). The branched tail ionizable lipids were named based on the number of carbons in the hydrophobic tail. The first number indicates the number of carbons, while the number in parentheses is the branching point, and the length of the branch is the last number (FIG. 1A, bottom). Linear ionizable lipids were named based on the number of carbons in the hydrophobic chain. In total, 22 lipids were synthesized (FIG. 1B). The branched tail lipid nanoparticle 306Oᵢ₁₀ was renamed 10(8)3 due to its branching structure (Hajj et al., supra; and Hajj et al., Nano Lett (2020), 5167-5175).

The library of BRAT ionizable lipids was generated and used to investigate how systematically changing the branching point on the hydrophobic chain of the lipid impacts delivery. Altering the branching point and branch length also changes the length of the longest lipid chain, leading to shorter hydrophobic lipid tails such as 10(1)4 and 10(2)4, whose longest hydrophobic chain length is 6 carbons, which is ineffective at mRNA delivery when by itself in a linear tail (Hajj et al. (2019), supra).

### Branched Tail Lipid Nanoparticle Characteristics are Consistent Across Chemistries.

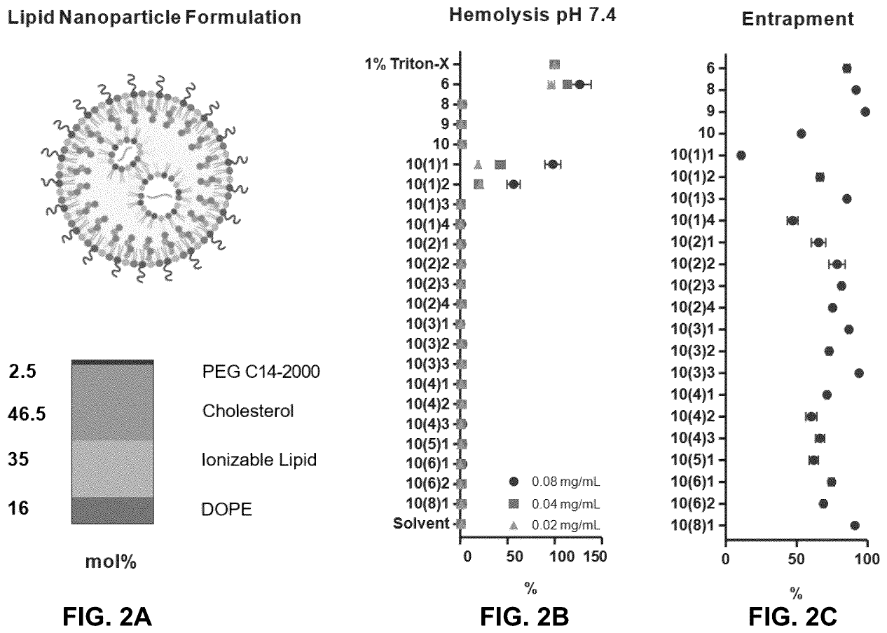

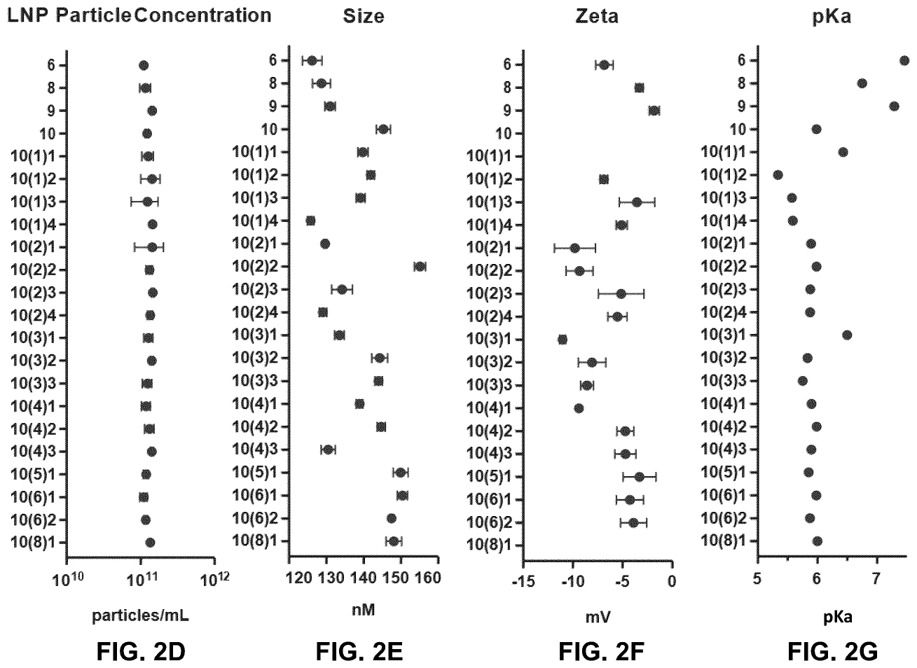

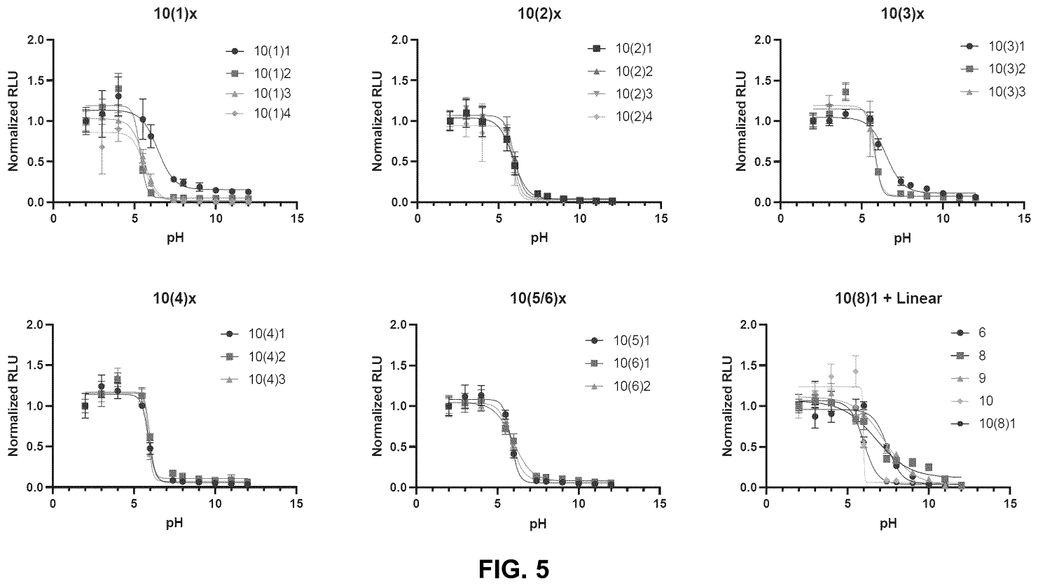

Lipid nanoparticles (LNPs) were formulated with PEG C14-2000, cholesterol, ionizable lipid, and DOPE at molar ratios of 2.5:46.5:35:16 and an mRNA to lipid ratio of 1:10(FIG. 2A). To fully characterize the BRAT lipid nanoparticle, several quality control tests were performed. Hemolysis of human red blood cells was conducted at pH 7.4 to determine whether lipid nanoparticles might exhibit cytotoxicity. Lipid nanoparticles 6, 10(1)1, and 10(1)2 demonstrated hemotoxicity (FIG. 2B), but none of the other lipid nanoparticles tested demonstrated hemotoxicity. Lipid nanoparticle entrapment efficiency was on average 70% (FIG. 2C). Lipid nanoparticle 10(1)1 had the lowest entrapment at 11%, while nine others had the highest entrapment at 99%. Entrapment of mRNA is important for effective delivery, but entrapment percentage is a poor predictor of in vivo efficacy (Hajj et al. (2019), supra). The concentration of lipid nanoparticles was determined using a Nanoparticle Tracking Analyzer to evaluate if BRAT lipid nanoparticles formed more nanoparticles/mL than their linear counterparts. There was no significant difference between the linear and branched tail nanoparticle concentrations (FIG. 2D). The concentration was found to range from 1.10×10¹¹ to 1.46×10¹¹ nanoparticles/mL, and averaged 1.30+0.12×10¹¹ nanoparticles/mL (FIG. 2D). The size of the lipid nanoparticles averaged about 139.3 nm, and ranged from 125.8 to 155.1 nm (FIG. 2E). Lipid nanoparticles having a size between 20 and 200 nm in diameter are robust enough to withstand the fluid flow while also being able to navigate through organs' interstitial space (Eygeris et al., supra). All lipid nanoparticles tested fell within this size range. The surface charge of the lipid nanoparticles was measured using the zeta potential. All ionizable lipids were slightly negatively charged at pH 7.4, with values ranging from −11.0 to −1.82 mV (FIG. 2F). The optimal pKa for lipid nanoparticles is thought to be about 6.1(Whitehead et al., supra; and Hajj et al. (2019), supra). The pKa for the lipid nanoparticles was determined by performing TNS fluorescence from pH 2 to 12(FIG. 5). For the lipid nanoparticles tested, the pKa ranged from 5.3 to 7.5, with an average of 6.1(FIG. 2G). Measurements of ionization using TNS were conducted at both pH 7.4(physiological) and 5.5(endosomal). Ionization values were higher for pH 5.5 in all lipid nanoparticles tested. Overall, the branched tail lipid nanoparticles were comparable to other lipid nanoparticles found in the literature relative to their size, entrapment, and charge (Hajj et al. (2019), supra; and Hashiba et al., Small Science (2023), 3:2200071).

Branched Tail Lipid Nanoparticles Potently Deliver mRNA In Vivo.

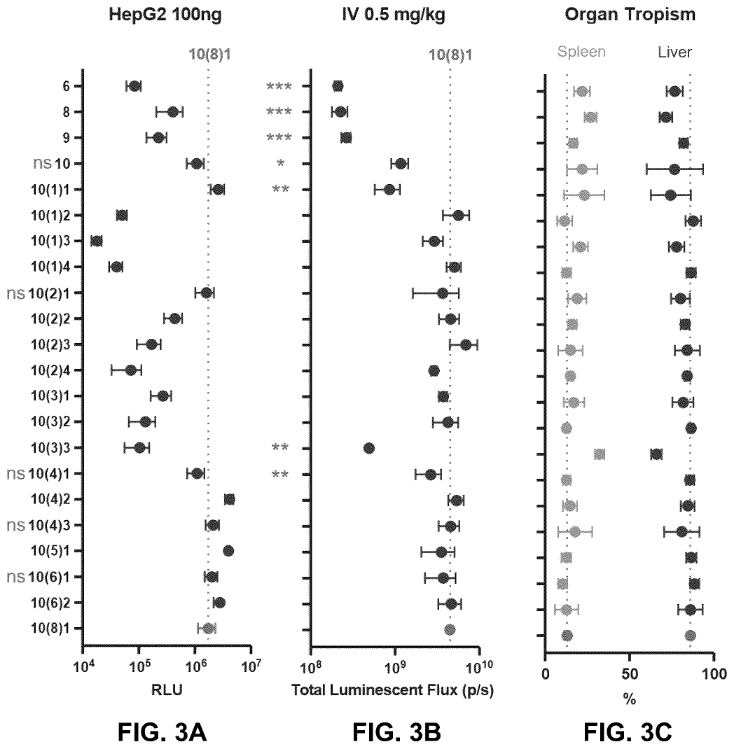

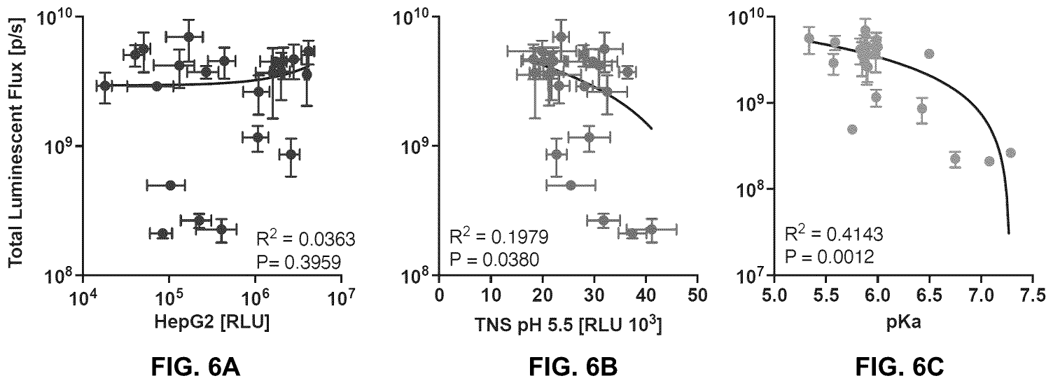

Branched tail lipid nanoparticles were screened in vitro by delivering 100 ng of luciferase mRNA to the immortalized human hepatocyte cell line HepG2. The branched tail ionizable lipid 10(8)1, also known as 306O i10, results in liver-specific lipid nanoparticles (Hajj et al. (2020), supra). Of the 22 lipid nanoparticles tested, 9 were as effective or better than 10(8)1 at mRNA delivery (FIG. 3A). However, work described elsewhere has indicated that in vitro screening of lipid nanoparticles does not always accurately predict the in vivo efficacy (Hajj et al. (2019), supra; and Paunovska et al., Nano Lett (2018), 18:2148-2157). Thus, all branched tail lipid nanoparticles were screened in vivo by delivering 0.5 mg/mL of luciferase mRNA intravenously to C57Bl/6 mice (FIG. 3B). The correlation between in vitro and in vivo efficacy was not significant, corroborating findings described elsewhere (Paunovska et al., supra; and Whitehead et al., ACS Nano (2012), 6:6922-6929) (FIG. 6A). Of the branched tail lipid nanoparticles tested, 88% were as potent as 10(8)1. Only 10(1)1 and 10(3)3, were less potent. Characterization of 10(1)1 via hemolysis (FIG. 2B) indicated it was hemolytic, which could contribute to its lower efficacy. The lower efficacy of 10(3)3 may be due to the point of branching in its lipid tail and the length of the branch. Both 10(2)4 and 10(4)3 contain short carbon chains of 6 and 7 carbons, respectively, but they have branching structures that are symmetrical. Research described elsewhere indicated that symmetry in the branching structure leads to more potent delivery, and these studies supported that finding (Hashiba et al., supra).

Branched tail lipid nanoparticle efficacy correlated with enhanced ionization at endosomal pH and pKa value (FIGS. 6B-6C). These findings were consistent with other findings (described elsewhere) that increased ionization at endosomal pH correlates with increased in vivo efficacy, and further demonstrated the importance of having branching in the ionizable lipid (Hajj et al. (2019), supra; and Hashiba et al., supra). The work described herein identified 15 new branched tail lipid nanoparticles that operate at the same level as the top-performing 10(8)1.

Organ tropism also was evaluated for all lipid nanoparticles tested in vivo (FIG. 3C). Delivery was primary detected in the liver and spleen, with 75% to 89% seen in the liver and 10% to 23% seen in the spleen for branched tails (excluding 10(3)3). Interestingly, while 10(3)3 bad lower efficacy than 10(8)1, it did have higher spleen expression (32% vs. 13%). Delivery specificity for linear tails 6-9 also saw shifts toward the spleen. Delivery outside the liver and spleen was about 1% for all lipid nanoparticles tested. Overall, organ tropism remained consistent across the lipid nanoparticle branched type. This may indicate that ionizable lipid head groups rather than tail groups drive tropism to other organs.

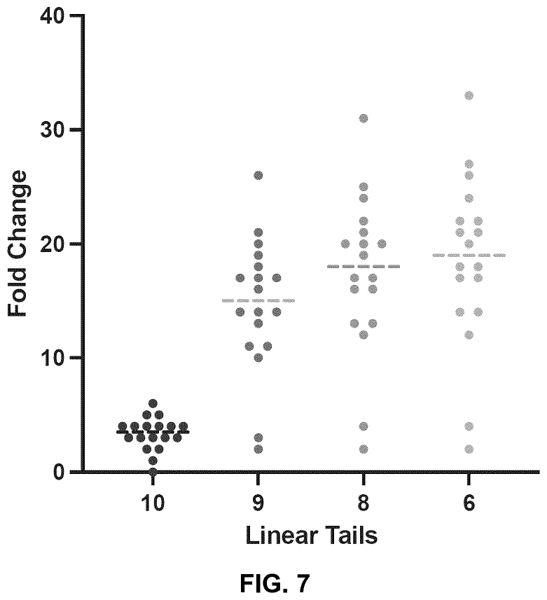

With 10 carbons total in the tail structure, branches that had a higher branch length had shorter overall lipid tail lengths. For example, the longest carbon chain for 10(1)4 and 10(2)4 is 6 carbons in length, but they outperformed 6 linear carbon by 24 and 14-fold, respectively. This trend continued for branches that had 8 and 9 carbons in the lipid chain. Even 10(3)3, which was significantly less potent than 10(8)1, outperformed its linear counterpart by 2-fold. Branched tail lipid nanoparticles with a longest carbon chain of 8 exhibited a 19- to 25-fold increase in delivery, while a longest chain of 9 carbons saw a 13- to 17-fold change (FIG. 7). This work indicated that branching in the tail structure results in potent delivery vehicles, regardless of the shorter hydrophobic carbon chain length.

### Branched Tail Lipid Nanoparticles are Versatile Delivery Vehicles.

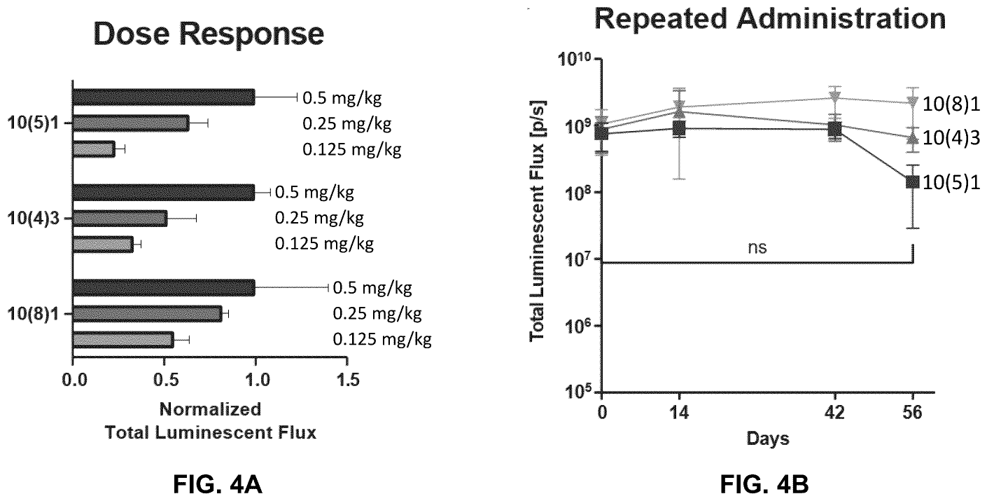

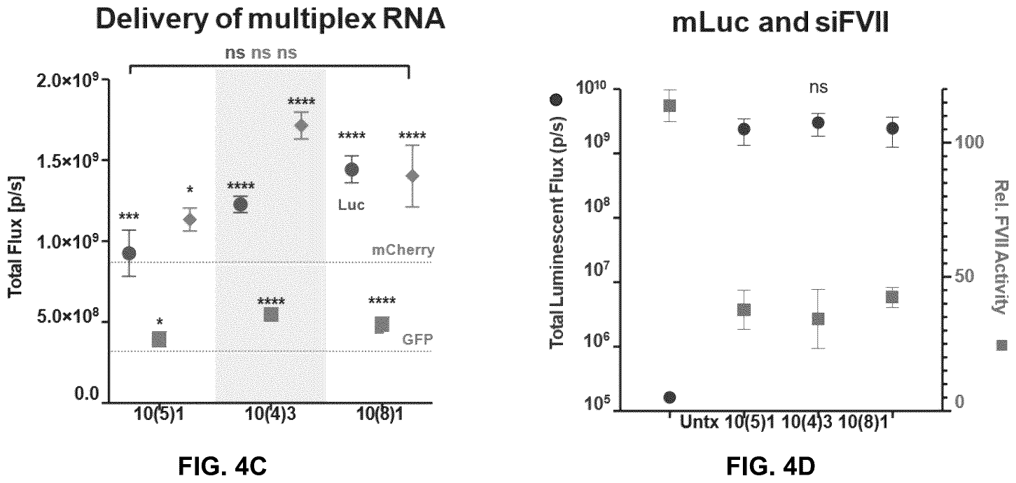

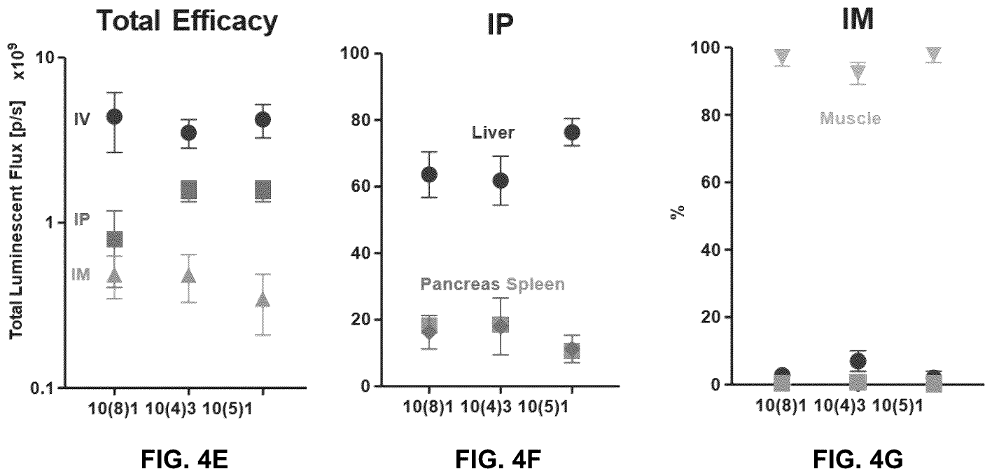

Studies were conducted to further explore the mRNA delivery capabilities of the BRAT ionizable lipids. Two branched tail lipid nanoparticles (10(5)1 and 10(4)3) were selected. Ionizable lipid 10(5)1 is similar to 10(8)1 due to its branch having one carbon, but the placement of the branch is further up the lipid chain. Ionizable lipid 10(4)3 has a shorter hydrophobic tail length, with seven carbons and a longer branch. Additionally, 10(4)3 is symmetric in its tail structure, with each lipid chain off the branched point being three carbons long. Research described elsewhere benchmarked 10(8)1 for efficacy and immunogenicity against previous “gold standards” C12-200 and MC3, the first ionizable lipid approved for siRNA delivery. Work described elsewhere found that 10(8)1 LNPs were well-tolerated upon intravenous delivery (Hajj et al. (2020), supra). Therefore, 10(8)1 was used as the benchmark for 10(5)1 and 10(4)3. Both 10(5)1 and 10(4)3 branched tail lipid nanoparticles behaved in a dosage-dependent manner when delivered intravenously (FIG. 4A). Many mRNA therapeutics will need repeated administration to enact a lasting therapeutic effect, and some lipid nanoparticles undergo taxiphlaxis after repeated administration (Besin et al., Immunohorizons (2019), 3:282-293). Therefore, the BRAT lipid nanoparticles were tested for their ability to maintain potency after repeated administration. All three branched tail lipid nanoparticles effectively delivered mRNA repetitively four times over 60 days (FIG. 4B), without significant loss of efficacy. These data demonstrated that 10(5)1 and 10(4)3 can be used for repeated administration of a therapeutic.

Given the excellent in vivo efficacy of 10(5)1 and 10(4)3, studies were conducted to explore the ability of the BRAT lipid nanoparticles to deliver multiple RNA cargos, and to ascertain whether the length of the carbon tails would impact their ability to confer efficacious delivery. Delivering multiple mRNAs is becoming a critical requirement of lipid nanoparticles, as researchers and clinicians seek to make multivalent or combination vaccines (Peng et al., Cell Rep (2022), 40(5): 111160; John et al., Vaccine (2018), 36:1689-1699; and Awasthi et al., Sci Immunol (2019), 4(39): eaaw7083), create cancer immunotherapies (Liu et al., supra), and treat disease (Melamed et al., Sci Adv (2023), 9(4), doi: 10.1126/sciadv.ade144). To assess the ability of the BRAT lipid nanoparticles to deliver diverse RNA cargo, formulated lipid nanoparticles were formulated with three mRNAs-mLuc, mGFP, and mmCherry (FIG. 4C). All branched tail lipid nanoparticles were able to effectively deliver the three multiplexed mRNAs. There was no significant difference between chemistries for any of the mRNAs tested, indicating that the different branching structures did not inhibit multiplexed mRNA, Delivering different types of RNA cargo together also can be beneficial. For example, a formulation may incorporate siRNA for VEGF to enhance chemosensitivity, while also delivering an mRNA for a tumor-specific antibody. Therefore, the ability of these branched tails to deliver both mRNA and siRNA together was assessed. Lipid nanoparticles were formulated at a total RNA concentration of 0.106 mg/kg and delivered at 0.5 mg/kg mLuc and 0.003 mg/kg siFVII, as that was identified elsewhere as an optimal formulation for co-delivery of mRNA and siRNA (Ball et al., Nano Lett (2018), 18:3814-3822). Delivery potency was the same across treatment groups for co-delivery of mRNA and siRNA (FIG. 4D). Together, these data demonstrated that BRAT lipid nanoparticles can deliver a diverse set of cargo.

Having demonstrated that these branched tail lipid nanoparticles can be dosed repetitively and can deliver diverse cargos, further studies were conducted to assess their delivery characteristics following administration by multiple injection routes. As described elsewhere, lipid nanoparticle efficacy and organ tropism are route-dependent (Besin et al., supra; Melamed et al., supra; and Hajj et al. (2020), supra). Accordingly, 10(5)1 and 10(4)3 were tested for efficacy following intraperitoneal and intramuscular injection (FIG. 4E). The total efficacy for each administration route was similar for all branched tails. Organ tropism shifted to the pancreas for intraperitoneal delivery (Melamed et al., supra) (15% of total signal) and to muscle tissue for intramuscular injection (Ci et al., Drug Met Disposit (2023), 51:813-823) (96% of total signal) (FIGS. 4F-4G). Overall, branched tail lipid nanoparticles efficiently delivered mRNA across administration routes, irrespective of the length or point of branching.

Taken together, these studies identified 15 new ionizable lipids for potent mRNA lipid nanoparticle delivery, and demonstrated that branching can enhance the potency of shorter ionizable lipid tails.

TABLE 1
Alcohol starting materials used to synthesize
branch tails for ionizable lipids.

| Alcohol | | |
|---|---|---|
| Starting Material | Name | Supplier |
| 10(1)1 | 2-decanol | TCI America |
| 10(1)2 | 3-decanol | Alfa Aesar |
| 10(1)3 | 4-decanol | Alfa Aesar |
| 10(1)4 | 5-decanol | TCI America |
| 10(2)1 | 2-methyl-1-nonanol | Jubilant Biosys |
| 10(2)2 | 2-ethyl-1-octanol | Jubilant Biosys |
| 10(2)3 | 2-propyl-1-heptanol | Toronto Research Chemicals |
| 10(2)4 | 2-tetra-1-hectanol | Jubilant Biosys |
| 10(3)1 | 3-methylnonan-1-ol | Jubilant Biosys |
| 10(3)2 | 3-ethyl-octan-1-ol | Jubilant Biosys |
| 10(3)3 | 3-propyl-1-heptanol | Jubilant Biosys |
| 10(4)1 | 4-methyl-1-nonanol | Jubilant Biosys |
| 10(4)2 | 4-ethyl-1-octanol | Jubilant Biosys |
| 10(4)3 | 4-propylheptan-1-ol | Jubilant Biosys |
| 10(5)1 | 5-methyl-1-nonanol | Jubilant Biosys |
| 10(6)1 | 6-methyl-1-nonanol | Jubilant Biosys |
| 10(6)2 | 6-ethyl-1-octanol | Jubilant Biosys |

TABLE 2
Branched tail ionizable lipids were confirmed to be fully
substituted via mass spectrometry. The expected mass for
lipidoids with 10 carbons in the lipid chain is 994.53.

| Electrospray | | | |
|---|---|---|---|
| M5 (+ve): | M | M/2 | M + Na |
| 306O₁₀₍₁₎₁ | 994.87 | 497.93 | |
| 306O₁₀₍₁₎₂ | 994.88 | 497.94 | |
| 306O₁₀₍₁₎₃ | 994.87 & 995.80 | | 1016.93 |
| 306O₁₀₍₁₎₄ | 994.87 & 995.80 | | 1016.87 |
| 306O₁₀₍₂₎₁ | 994.87 | 498.00 | |
| 306O₁₀₍₂₎₂ | 994.87 | 497.93 | |
| 306O₁₀₍₂₎₃ | 994.88 | 497.94 | |
| 306O₁₀₍₂₎₄ | 994.88 | 497.94 | |
| 306O₁₀₍₃₎₁ | 994.87 | | |
| 306O₁₀₍₃₎₂ | 994.88 | 497.94 | |
| 306O₁₀₍₃₎₃ | 994.88 | 497.94 | |
| 306O₁₀₍₄₎₁ | 994.93 | 497.93 | |
| 306O₁₀₍₄₎₂ | 994.88 | 497.94 | |
| 306O₁₀₍₄₎₃ | 994.88 | 497.94 | |
| 306O₁₀₍₅₎₁ | 994.87 | | |
| 306O₁₀₍₆₎₁ | 994.87 | | |
| 306O₁₀₍₆₎₂ | 994.87 | | |

### Example 2—Branch Tail Lipid Nanoparticles with 11 Carbons Effectively Co-Deliver Three Multiplexed RNAs

### Methods and Materials

Synthesis of Lipidoid Tails: Branched tails for the lipidoids were synthesized by reacting alcohols (TABLE 3) with acryloyl chloride (Alfa Acsar) and trimethylamine (Sigma Aldrich) in a molar ratio of 1:1.5:2 in reagent grade acetone (Spectrum) in a round bottom flask on ice. Ice was removed after about ten minutes, and the flask was allowed to equilibrate to room temperature for two hours. The reaction was quenched with 3 mL of deionized water for ten minutes. The product was then rotary evaporated for about 1.5 hours and then dissolved in ethyl acetate and placed in a separation funnel. Four washes were done to remove contaminants: (1) NaCl (saturated) and water in a 1:1 molar ratio, (2)1N HCl and water in a 1:1 molar ratio, (3) NaHCO₃(saturated), and (4) NaCl (saturated). The product was then retrieved and 3-6 mg of 2,5 di-tert-butylhydroquinone (Sigma Aldrich) was added to prevent polymerization. Magnesium sulfate (Fisher Chemicals) was then added to remove water, and was then filtered out. The product was rotary evaporated to remove the solvent. The tail was then purified using flash chromatography on an ISCO CombiFlash system equipped with a silica column (solid-phase silica). The sample was loaded onto the column pre-equilibrated with DCM. Elution was performed using a gradient of DCM: methanol: ammonium hydroxide (60:30:10, v/v/v) that did not exceed 50%.

Synthesis of Lipidoids: As described elsewhere (Whitehead et al. (2014), supra), lipidoids were synthesized by the addition of alky-acrylates to amines. Amine 3,3′-diamino-N-methyldipropylamine (306, Sigma Aldrich), was reacted with custom synthesized alky-acrylates of different lengths and branching. Acrylates were combined with amines at a molar ratio of 4:1 at 90° C. for 3-7 days in a glass scintillation vial. Fully substituted lipids were confirmed with mass spectrometry (TABLE 4).

Formation of Lipid Nanoparticles: LNPs were formulated by mixing lipidoid, cholesterol (Sigma Aldrich), 1,2-dioleoyl-sn-glycero-3-phosphoethanolamine (DOPE, Avanti Polar Lipids), and C14-PEG2000(Avanti Polar Lipids) in a solution of ethanol and 10 mM sodium citrate at a molar ratio of 35:46.5:16:2.5. mRNA was diluted in 10 mM sodium citrate buffer. Lipidoid and mRNA mixtures were combined and then diluted to the desired concentration using phosphate-buffered saline. Lipid nanoparticles had a final weight ratio of 1:10 mRNA to lipidoid. For in vivo experiments, all lipid nanoparticles were dialyzed against PBS pH 7.4 for at least one hour.

Malvern Zeta Sizer: Nanoparticle size and zeta potential were determined by dynamic light scattering (Malvern Zetasizer Nano ZSP). Lipid nanoparticles were diluted 1:100 from 0.005 mg/mL for testing. Three technical replicates were conducted on each sample for both size and surface zeta potential.

TNS Ionization and pKa values: TNS assays were conducted as described elsewhere (Hajj et al. (2019), supra) to evaluate ionization of the lipid nanoparticles. Briefly, 250 μL of a buffer consisting of 150 mM sodium chloride, 20 mM sodium phosphate, 20 mM ammonium acetate, and 25 mM ammonium citrate was added to each well. Then 10 μL of 0.16 mM stock solution of 2-(p-toluidinyl) naphthalene-6-sulphonic acid (TNS, Sigma Aldrich) in DI water was added along with 5 μL of 0.05 mg/mL mRNA lipid nanoparticles. Fluorescence intensity was measured at an excitation of 322 nM and an emission of 431 nm (Tecan Spark). The pKa of lipid nanoparticles was calculated by performing the TNS assay at pH 2 to 12 in increments of 1. A pH of 7.4 was used instead of 7 since pH 7.4 approximates physiological blood pH, and a pH of 5.5 was used instead of 5 since pH 5 approximates the late-stage endosomal pH. mRNA used for these experiments was mLuc from Translate Bio.

Entrapment: To measure RNA entrapment, intact and lysed nanoparticles were measured for RNA content using a Quant-iT™ RiboGreen™ RNA Assay Kit according to the manufacturer's instructions. Lipid nanoparticles were synthesized at a concentration of 0.005 mg/mL (firefly luciferase) and then diluted ten-fold. Briefly, LNPs were diluted in equal volumes of Tris-EDTA buffer or 2% Triton X-100 in DI water buffer. An equal volume of RiboGreen™ reagent was then added to each sample and incubated at 37° C. for 15 minutes. The fluorescence was read on a Tecan Spark® Multimode Microplate Reader. The excitation signal was 480 nm and emission 520 nm.

In Vivo Testing of Lipid Nanoparticles: Female C57BL/6 mice about 6 weeks in age were used. Lipid nanoparticles were intravenously injected via the tail vein at a concentration of 0.5 mg/kg of mRNA encoding firefly luciferase. About 3 hours after injection, 130 μL of d-luciferin (PerkinElmer) was administered intraperitoneally at a concentration of 30 mg/mL. Fifteen minutes after the d-luciferin injection, the mice were sacrificed and their organs were imaged using the In Vivo Imaging Instruments (IVIS).

Multiplex RNA Delivery: Female and male Ai9 mice about 6 weeks of age were used. Lipid nanoparticles were intravenously injected via the tail vein at a concentration of 0.33 mg/kg firefly luciferase mRNA, 0.33 mg/kg Cre Recombinase mRNA, and 0.006 mg/kg siRNA for Factor VII. About 6 hours after injection of the lipid nanoparticles, 130 μL of d-luciferin (PerkinElmer) was administered intraperitoneally at a concentration of 30 mg/mL. Fifteen minutes after the intraperitoneal injection, the mice were live imaged using the IVIS for luciferase signal. At 48 hours, the mice were live imaged for tdTomato expression (550/580) and submandibularly bled for Factor VII. For Factor VII detection, a BIOPHEN FVII assay was used according to the manufacturer's protocol (Aniara, OH).

Statistical Analysis: Living Image software was used to analyze data. All statistical analysis was conducted using GraphPad Prism (La Jolla, CA). Standard deviation is abbreviated as “SD” in the figure descriptions. Significance was determined by Tukey's multiple comparisons test.

### Results

### Synthesis of a Small Library of Branched Tail Lipid Nanoparticles.

Given the increased potency associated with branched ionizable lipid tails, a library of branched-tail ionizable lipids containing an 11-carbon hydrophobic chain was synthesized to investigate their potential for enhancing lipid nanoparticle potency. Branched tails were synthesized by converting alcohols to acrylates (FIG. 1A). After purification, branched tails were combined with amine-containing head group 306 via a Michael addition reaction for about 3 days at 90° C., yielding a branched tail ionizable lipid (FIG. 1B). Mass spectrometry confirmed that the ionizable lipids were fully substituted and four-tailed (TABLE 4). Seven unique ionizable lipids were synthesized in total (FIG. 8). Of these seven lipids, four had a branch off the first carbon, with different tail lengths. The other three lipids had branches at the end of the carbon chain. End-tail carbon chain branches can increase delivery efficacy of ionizable lipids 306O₁₀ and 306Oᵢ₁₀

(Hajj et al. (2019), supra). The branched tails were named for the number of carbons in the lipid tail, followed by the branching point in parentheses, and the length of the branch. Notably, 11(1)5, 11(7)2, and 11(9)1 all contained symmetrical branches can increase delivery efficacy (Hashiba et al., supra).

### Branched Tail Lipid Nanoparticles Pass Quality Control Tests.

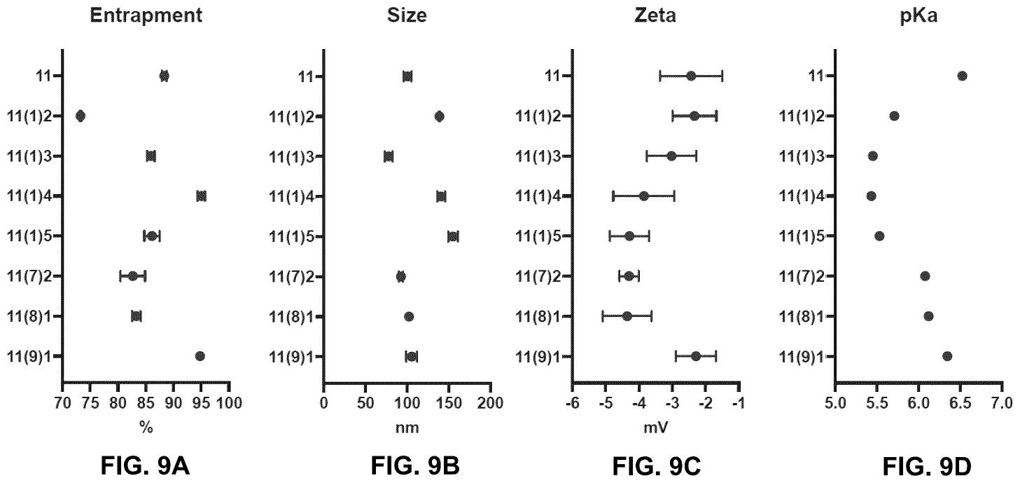

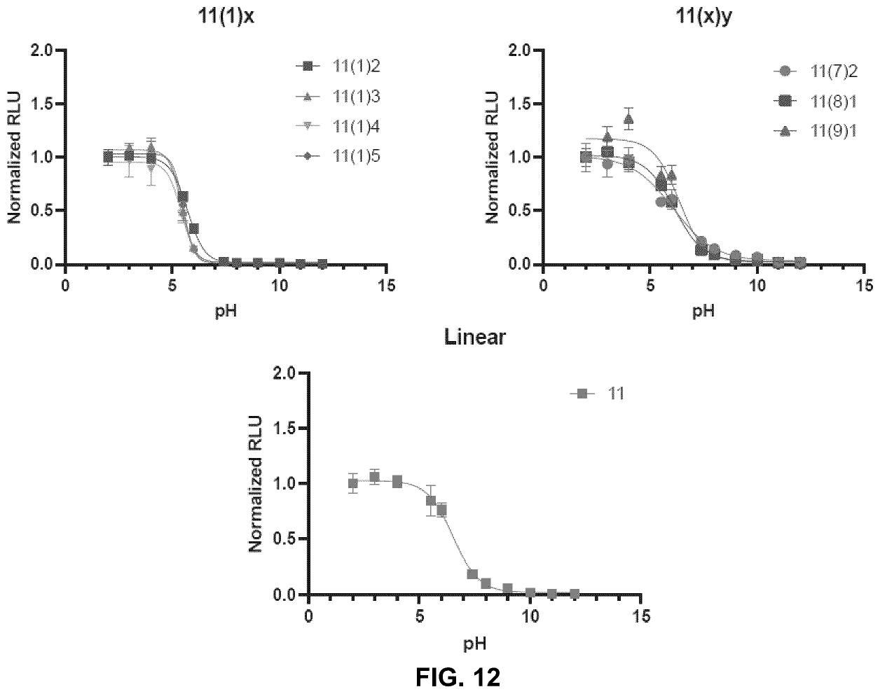

Before screening the lipid nanoparticles in vivo, quality control tests were conducted. The ability to entrap the mRNA cargo is important, as entrapment protects the mRNA from degradation and prevents activation of TLR4 pathways that naked mRNA can activate (Hajj and Whitehead, supra). A general and arbitrary cut-off for mRNA lipid nanoparticle entrapment is >70%, although mRNA entrapment does not correlate with in vivo efficacy (Hajj et al. (2019), supra). Nonetheless, encapsulating as much RNA possible was thought to be beneficial for downstream processing of the future product and to conserve material cost. All branched tail lipid nanoparticles had an mRNA entrapment above 70%, and on average entrapment was 86% (FIG. 9A), Next, the size of the lipid nanoparticles was characterized and all were found to be below 155 nm (FIG. 9B). Next, the surface charge of the branched tail lipid nanoparticles was measured at a physiological pH of 7.4. For all lipid nanoparticles tested, the zeta potential was negative (FIG. 9C). Most lipid nanoparticles exhibit a slight negative charge at neutral pH. For ionizable lipid nanoparticles at lower pH (such as pH 5.5 that mimics the late endosomes), the surface charge drops. Studies of protein corona formation revealed that negative zeta potential can attract a large number of proteins due to electrostatic interactions with cationic patches of proteins found in the blood (Bilardo et al., Wiley Interdiscip Rev Nanomed Nanobiotechnol (2022), 14(4): e1788). The pKa of the lipid nanoparticles was measured by measuring ionization using the TNS assay with buffers having pH from 2-12(FIG. 12). The pKa was found to be 5.9 on average. Linear carbon tail 11 had the highest pKa at 6.5, while 11(1)5 had the lowest at 5.3(FIG. 9D). pKa typically correlates with in vivo efficacy, with an optimal pKa of about 6.1. Overall, these studies demonstrated that branched tail lipid nanoparticles with 11 carbons were comparable to other ionizable lipids described elsewhere (Hajj et al. (2019), supra; and Lokugamage et al., Adv Mat (2019), 31:1902251), and passed quality control tests to move into in vivo studies.

### Branched Tail Lipid Nanoparticles can Outperform Linear Carbon Counterparts.

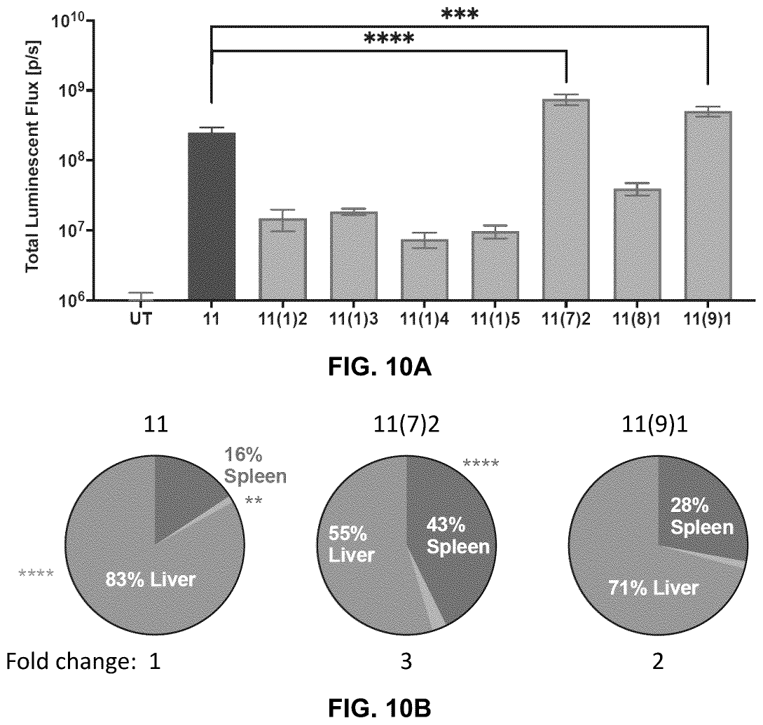

Branched tail lipid nanoparticles were delivered intravenously to C57BL/6 mice at 0.5 mg/kg of mRNA for firefly luciferase. Untreated and linear carbon chain 11 controls also were tested. Two of the seven-branched tail lipid nanoparticles were found to be more potent than the 11-carbon linear tail (FIG. 10A). In particular, branched tail lipid nanoparticle 11(7)2 was 3-fold more potent, while 11(9)1 was 2-fold more potent. These two branched tail lipid nanoparticles contain symmetrical branches at the end of the carbon chain, similar to that of 306Quo. When the branched point occurred on the first carbon, however, the branched tail lipid nanoparticles did not convey efficient mRNA delivery. These nanoparticles had the lowest pKa's, ranging from 5.3 to 5.7, which is lower than the favored 6.1375, further highlighting the “sweet spot” for the pKa of the lipid nanoparticle to convey potent mRNA delivery.

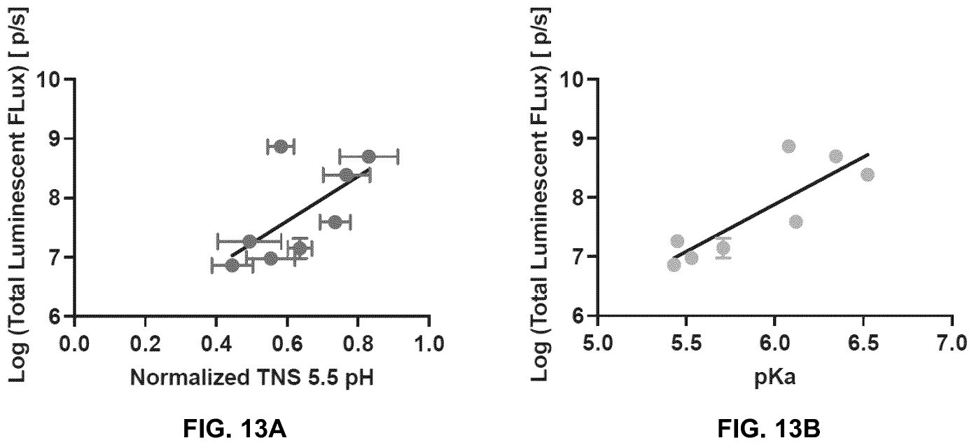

Branched tail lipid nanoparticles may exhibit increased efficacy due to enhanced ionization at endosomal pH (Hajj et al. (2019), supra). Studies described elsewhere confirmed that symmetrically branched tail lipid nanoparticles demonstrate higher efficacy and greater ionization at endosomal pH 5.5. The studies described herein revealed a significant positive non-zero slope between normalized ionization at pH 5.5, although the Pearson correlation was not statistically significant (P=0.0887) (FIG. 13A). Taken together, the non-zero slope and the P-value close to 0.05 suggested that higher ionization at endosomal pHI may be predictive of in vivo LNP efficacy. Interestingly, the relationship between in vivo efficacy and pKa showed a significant positive correlation with a non-zero slope (FIG. 13B). It is possible that branching at the first carbon impacted the packing of the lipid nanoparticle and, thus, the ionization capabilities. The packing may be more spaced due to the higher branching point, creating more space around the head group, although molecular modeling would be needed to understand if this phenomenon would occur. However, such a change in packing may also lead to less stable lipid nanoparticles in complex biological fluids. While a change in the packing due to branching at the first carbon may explain the lower efficacy of 11(1)2 to 11(1)5 lipid nanoparticles, 11(8)1 features branching at the end of the carbon, forming an anti-iso branched tail. Anti-iso-branched fatty acids typically tare more effective at fluidizing membranes (Frank et al., J Biol Chem (2021), 297(5): 101255) than iso-branched fatty acids, potentially facilitating cellular entry and endosomal escape of LNPs. However, excessive fluidization may induce toxic effects, thereby reducing mRNA lipid nanoparticle delivery. Moreover, 11(8)1 lacks symmetry in its branched structure.

Organ tropism was also of interest for lipid nanoparticle delivery, as an important use of lipid nanoparticles is to treat diseases and create prophylactics. Among the most potent branched tail lipid nanoparticles, there was a significant shift in liver and spleen expression for 11(7)2 and 11(9)1 compared to the linear 11 lipid nanoparticle (FIG. 10B). Spleen tropism was also significantly higher for 11(7)2 than 11(9)1. Specific delivery to the spleen is useful for therapies that aim to be immunomodulators or for treating immune-related diseases.

### Branched Tail Lipid Nanoparticles Effectively Deliver Three Multiplexed RNAs.

Combination therapies can be used to treat cancer and infectious diseases (Gilad et al., Cancers (Basel) (2021), 13:1-26), as they can combine therapies that target disease via different mechanisms. Delivery of mRNA leads to protein production while delivery of siRNA downregulates protein production, providing mechanistically different impacts on protein expression. Co-formulation of mRNA and siRNA has been demonstrated for one mRNA and one siRNA (Ball et al., supra), but treatment of infectious diseases with antibodies would require the successful delivery of two mRNAs-one for the heavy chain and another for the light chain. Antibodies have been successfully delivered for treatment of diseases such as COVID-19(Deng et al., Cell Res (2022), 32:375-382) and Rift Valley Fever Virus (Wang et al., supra), and delivery of bispecific T-cell engaging (BiTE) antibodies have been used for cancer treatment (Huang et al., Adv Sci (2023), 10:2205532). Antibodies produced by mRNA can identify infected cells and mark them for targeting and clearance by the immune system. On the other hand, siRNAs that specifically silence viral RNAs can halt viral replication in the cytoplasm, thereby lowering the viral load (Mehta et al., Adv Healthc Mater (2021), 10(7): 2001650). To determine whether these approaches can be combined, studies were conducted to determine whether packaging two mRNAs and an siRNA together was possible with the lipid nanoparticles described herein.

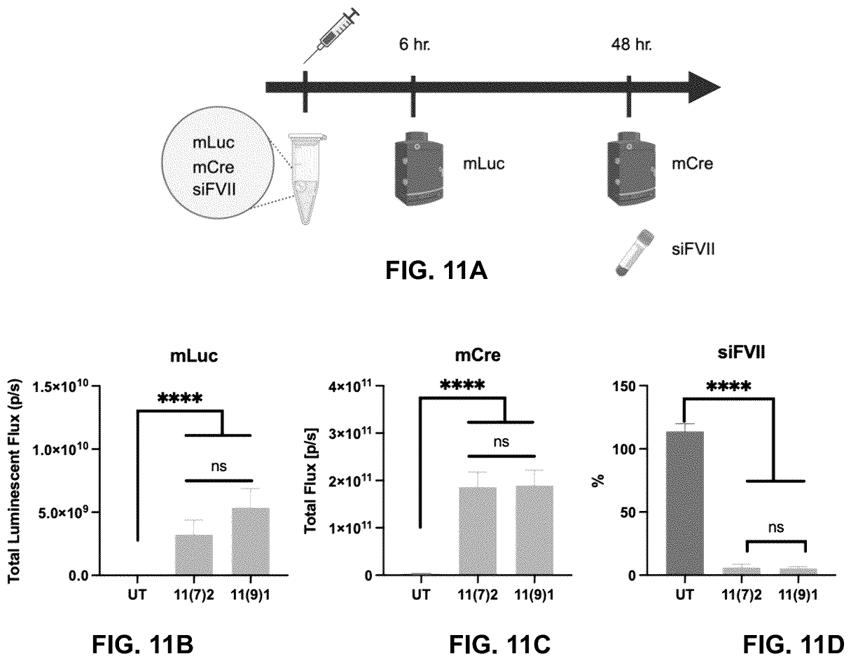

The heavy chain and light chain of antibodies can vary in size. A study reporting a size of 2.0 kb for the heavy chain and 1.5 kb for the light chain and was used as a guide for reporter mRNA selection (Wang et al., Mol Pharm (2024), 21:1342-1352). Cre recombinase (1.4 kb) and firefly luciferase (1.9 kb) were selected as the reporter mRNAs that would mimic the size of heavy and light chain mRNAs. For siRNA, Factor VII was selected for its ease of detection; the size of siRNA does not change drastically (Whitehead et al., Nat Rev Drug Discov (2009), 8:129-138). Ai9 mice were injected intravenously with co-formulated lipid nanoparticles containing 0.33 mg/kg of each mRNA and 0.006 mg/kg of siRNA. Branched tail ionizable lipids 11(7)2 and 11(9)1 were each tested and compared to an untreated control. Luciferase expression was 5 measured at 6 hours, while tdTomato and Factor VII were measured at 48 hours (FIG. 11A). Both branched tail lipid nanoparticles were able to induce protein expression and Factor VII knockdown (FIGS. 11B-11D), demonstrating successful co-delivery of two mRNAs approximately the same size of an antibody together with a siRNA.

TABLE 3
List of starting materials for 11 carbon branch tails.

| | | | Custom |
|---|---|---|---|
| Abbreviation | Alcohol Name | Purchased | (Y/N) |
| 11(1)2 | 3-Undecanol | TCI America | N |
| 11(1)3 | 4-Undecanol | TCI America | N |
| 11(1)4 | 5-Undecanol | TCI America | N |
| 11(1)5 | 6-Undecanol | TCI America | N |
| 11(7)2 | 7-ethylonan-1-ol | Jubilant Biosys | Y |
| 11(8)1 | 8-Methy-1-decanol | AliChem | N |
| 11(9)1 | 9-Methyldecanol | Toronto Research Chem | N |

TABLE 4
Mass spectrometry data used to confirm substitution of amine-
containing head group with branch tails. The expected mass
for lipidoids with 11 carbons is 1050.69 g/mol.

| Electrospray | | | |
|---|---|---|---|
| MS (+ve): | M | M/2 | M + Na |
| 306O₁₁₍₁₎₂ | 1050.87 | | |
| 306O₁₁₍₁₎₃ | 1050.93 | 526.33 | 1072.93 |
| 306O₁₁₍₁₎₄ | 1050.87 | | 1072.93 |
| 306O₁₁₍₁₎₅ | 1050.93 | | 1072.87 |
| 306O₁₁₍₇₎₂ | 1050.93 | | |
| 306O₁₁₍₈₎₁ | 1050.93 | | |
| 306O₁₁₍₉₎₁ | 1050.93 | 526.00 | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A lipid-containing particle comprising:
a lipidoid;
cholesterol or a derivative thereof;
a helper lipid; and
a polyethylene glycol (PEG)-based compound,
wherein said lipidoid comprises an amine-containing head and one or more acrylate tails, wherein each of said one or more acrylate tails comprises an alkyl chain of 10 or 11 carbon atoms, and wherein said alkyl chain of each of said one or more acrylate tails comprises a one, two, three, four, or five carbon atom branch.

2. The lipid-containing particle of claim 1, wherein said lipidoid has two, three, or four acrylate tails.

3. The lipid-containing particle of claim 1, wherein said lipid-containing particle is a lipid nanoparticle (LNP).

4. The lipid-containing particle of claim 1, wherein said helper lipid is a neutral lipid or a zwitterionic lipid.

5. The lipid-containing particle of claim 1, wherein said lipidoid comprises an N¹-(3-aminopropyl)-N¹-methylpropane-1,3-diamine head.

6. The lipid-containing particle of claim 1, wherein said lipidoid comprises:
a decan-2-yl acrylate tail,
a decan-3-yl acrylate tail,
a decan-4-yl acrylate tail,
a decan-5-yl acrylate tail,
a 2-methylnonyl acrylate tail,
a 2-ethyloctyl acrylate tail,
a 2-propylheptyl acrylate tail,
a 2-butylhexyl acrylate tail,
a 3-methylnonyl acrylate tail,
a 3-ethyloctyl acrylate tail,
a 3-propylheptyl acrylate tail,
a 4-methylnonyl acrylate tail,
a 4-ethyloctyl acrylate tail,
a 4-propylheptyl acrylate tail,
a 5-methynonyl acrylate tail,
a 6-methylnonyl acrylate tail,
a 6-ethyloctyl acrylate tail,
an undecan-3-yl acrylate tail,
an undecan-4-yl acrylate tail,
an undecan-5-yl acrylate tail,
an undecan-6-yl acrylate tail,
a 7-ethylnonyl acrylate tail,
an 8-methyldecyl acrylate tail, or
a 9-methyldecyl acrylate tail.

7. The lipid-containing particle of claim 1, wherein the cholesterol or a derivative thereof is cholesterol.

8. The lipid-containing particle of claim 1, wherein the PEG-based compound is a PEG-lipid, and wherein the PEG has a molecular weight of about 300 g/mol to about 5000 g/mol.

9. The lipid-containing particle of claim 1, wherein one or more of the following is true:
at pH 5.5, said lipid-containing particle has a net positive charge; said lipid-containing particle has a diameter of about 60 nM to about 160 nM;
said lipid containing particle has a zeta potential at pH 7.4 of about −11 mV to about-2 mV; and
said lipid-containing particle has a pKa of about 5.3 to about 7.5.

10. A composition comprising the lipid-containing particle of claim 1, wherein said lipid-containing particle further comprises a therapeutic agent.

11. The composition of claim 10, wherein the therapeutic agent comprises RNA.

12. The composition of claim 11, wherein said RNA comprises messenger RNA (mRNA), short interfering RNA (siRNA), short hairpin RNA (shRNA), micro RNA (miRNA), antisense RNA, guide RNA, long non-coding RNA, transfer RNA, ribosomal RNA, double-stranded RNA (dsRNA), RNA aptamer, or any combination thereof.

13. The composition of claim 10, wherein said lipidoid comprises an N¹-(3-aminopropyl)-N¹-methylpropane-1,3-diamine head and a decan-2-yl acrylate tail, a decan-3-yl acrylate tail, a decan-4-yl acrylate tail, a decan-5-yl acrylate tail, a 2-methylnonyl acrylate tail, a 2-ethyloctyl acrylate tail, a 2-propylheptyl acrylate tail, a 2-butylhexyl acrylate tail, a 3-methylnonyl acrylate tail, a 3-ethyloctyl acrylate tail, a 3-propylheptyl acrylate tail, a 4-methylnonyl acrylate tail, a 4-ethyloctyl acrylate tail, a 4-propylheptyl acrylate tail, a 5-methynonyl acrylate tail, a 6-methylnonyl acrylate tail, a 6-ethyloctyl acrylate tail, an undecan-3-yl acrylate tail, an undecan-4-yl acrylate tail, an undecan-5-yl acrylate tail, an undecan-6-yl acrylate tail, a 7-ethylnonyl acrylate tail, an 8-methyldecyl acrylate tail, or a 9-methyldecyl acrylate tail.

14. A method for delivering a therapeutic agent to a mammal, wherein the method comprises administering to the mammal a composition comprising the lipid-containing particle of claim 1, wherein said lipid-containing particle further comprises said therapeutic agent.

15. The method of claim 14, wherein said therapeutic agent comprises RNA.

16. The method of claim 15, wherein said RNA comprises mRNA, siRNA, shRNA, miRNA, antisense RNA, guide RNA, long non-coding RNA, transfer RNA, ribosomal RNA, dsRNA, RNA aptamer, or any combination thereof.

17. The method of claim 14, wherein said lipidoid comprises:
(a) an N¹-(3-aminopropyl)-N¹-methylpropane-1,3-diamine head; and
(b) a decan-2-yl acrylate tail, a decan-3-yl acrylate tail, a decan-4-yl acrylate tail, a decan-5-yl acrylate tail, a 2-methylnonyl acrylate tail, a 2-ethyloctyl acrylate tail, a 2-propylheptyl acrylate tail, a 2-butylhexyl acrylate tail, a 3-methylnonyl acrylate tail, a 3-ethyloctyl acrylate tail, a 3-propylheptyl acrylate tail, a 4-methylnonyl acrylate tail, a 4-ethyloctyl acrylate tail, a 4-propylheptyl acrylate tail, a 5-methynonyl acrylate tail, a 6-methylnonyl acrylate tail, a 6-ethyloctyl acrylate tail, an undecan-3-yl acrylate tail, an undecan-4-yl acrylate tail, an undecan-5-yl acrylate tail, an undecan-6-yl acrylate tail, a 7-ethylnonyl acrylate tail, an 8-methyldecyl acrylate tail, or a 9-methyldecyl acrylate tail.

18. A lipid-containing particle comprising:
a lipidoid;
cholesterol or a derivative thereof;
a helper lipid; and
a polyethylene glycol (PEG)-based compound,
wherein said lipidoid comprises an amine-containing head and one or more acrylate tails, wherein said amine-containing head is N¹-(3-aminopropyl)-N¹-methylpropane-1,3-diamine, and wherein said one or more acrylate tails comprise one or more of a decan-3-yl acrylate tail, a decan-5-yl acrylate tail, a 2-methylnonyl acrylate tail, a 2-ethyloctyl acrylate tail, a 2-propylheptyl acrylate tail, a 3-ethyloctyl acrylate tail, a 4-ethyloctyl acrylate tail, a 4-propylheptyl acrylate tail, a 5-methynonyl acrylate tail, a 6-ethyloctyl acrylate tail, an undecan-3-yl acrylate tail, an undecan-4-yl acrylate tail, an undecan-5-yl acrylate tail, an undecan-6-yl acrylate tail, a 7-ethylnonyl acrylate tail, an 8-methyldecyl acrylate tail, and a 9-methyldecyl acrylate tail.

19. A composition comprising the lipid-containing particle of claim 18, wherein said lipid-containing particle further comprises a therapeutic agent.

20. A method for delivering a therapeutic agent to a mammal, wherein the method comprises administering to the mammal a composition comprising the lipid-containing particle of claim 18, wherein said lipid-containing particle further comprises said therapeutic agent.
